(19) United States
(12) Patent Application Publication
(10) Pub. No.: US 2022/0194935 A1
(43) Pub. Date: Jun. 23, 2022

(54) PHENETHYL SUBSTITUTED IMIDAZO[4,5-C]QUINOLINE COMPOUNDS WITH AN N-1 BRANCHED GROUP

(71) Applicant: 3M INNOVATIVE PROPERTIES COMPANY, St. Paul, MN (US)

(72) Inventors: George W. Griesgraber, Eagan, MN (US); Hannah C. Cohen, St. Paul, MN (US); Devon M. Hunerdosse, St. Paul, MN (US); Kevin J. Bechtold, St. Paul, MN (US)

(21) Appl. No.: 17/594,617

(22) PCT Filed: Jun. 3, 2020
(86) PCT No.: PCT/IB2020/055235

§ 371 Date: Oct. 25, 2021

Related U.S. Application Data
(60) Provisional application No. 62/860,543, filed on Jun. 12, 2019.

(51) Int. Cl. C07D 471/04

(52) U.S. Cl. CPC C07D 471/04

(57) ABSTRACT

Imidazo[4,5-c]quinoline compounds having a substituent that is attached at the N-1 position by a branched group, single enantiomers of the compounds, pharmaceutical compositions containing the compounds, and methods of making the compounds are disclosed. Methods of use of the compounds as immune response modifiers, for inducing cytokine biosynthesis in humans and animals, and in the treatment of diseases including infectious and neoplastic diseases are also disclosed.

### BACKGROUND

Some drug compounds act by stimulating certain key aspects of the immune system, as well as by suppressing certain other aspects (e.g., U.S. Pat. No. 6,039,969 (Tomai et al.) and U.S. Pat. No. 6,200,592 (Tomai et al.)). These compounds are sometimes referred to as immune response modifiers (IRMs). Some IRM compounds are useful for treating viral diseases, neoplasias, and T_{H}2-mediated diseases. Some IRM compounds are useful as vaccine adjuvants.

IRM compounds have been reported based on the following bicyclic and tricyclic ring systems: 1H-imidazo[4,5-c]quinolin-4-amines (e.g., U.S. Pat. No. 4,689,338 (Gerster)); 1H-imidazo[4,5-c]pyridin-4-amines (e.g., U.S. Pat. No. 5,446,153 (Lindstrom et al.)); 1H-imidazo[4,5-c][1,5]naphthyidin-4-amines (e.g., U.S. Pat. No. 6,194,425 (Gerster et al.)); thiazolo[4,5-c]quinolone-4-amines and oxazolo[4,5-c]quinolone-4-amines (e.g., U.S. Pat. No. 6,110,929 (Gerster et al.)); 6,7,8,9-1H-tetrahydro-1H-imidazo[4,5-c]quinolin-4-amines (e.g., U.S. Pat. No. 5,352,784 (Nikolaides et al.)); 2H-pyrazolo[3,4-c]quinolone-4-amines (e.g., U.S. Pat. No. 7,544,697 (Hays et al.)); and N-1 and 2-substituted 1H-imidazo[4,5-c]quinolin-4-amines (e.g., U.S. Pat. No. 6,331,539 (Crooks et al.), U.S. Pat. No. 6,451,810 (Coleman et al.), U.S. Pat. No. 6,664,264 (Dellaria et al.), U.S. Pat. No. 8,691,837 (Krepski et al.), U.S. Pat. No. 8,088,790 (Kshirsagar et al.), U.S. Pat. No. 8,673,932 (Kshirsagar et al.), U.S. Pat. No. 8,697,873 (Krepski et al.), and U.S. Pat. No. 7,915,281 (Krepski et al.)).

### SUMMARY

New compounds that can be useful in inducing cytokine biosynthesis in humans and animals are disclosed. Such compounds (or salts thereof) are of the following Formula (I):

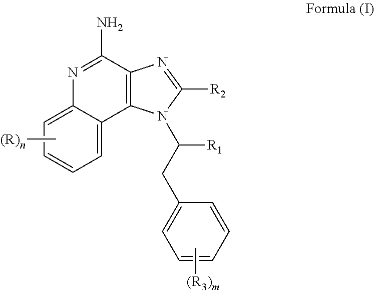

wherein:

m is an integer of 0 or 1;

n is an integer of 0 or 1;

R is selected from the group consisting of halogen, hydroxy, alkyl, alkoxy, and —C(O)—O-alkyl;

R₁ is alkyl or —CH₂—O—C₁₋₄alkyl;

R₂ is selected from the group consisting of hydrogen, methyl, ethyl, n-propyl, n-butyl, —CH₂OCH₃, —CH₂OCH₂CH₃, and —CH₂CH₂OCH₃; and

R₃ is selected from the group consisting of halogen, hydroxy, alkyl, and alkoxy; with the proviso that when R₃ is alkoxy, R₁ is alkyl.

The compounds of Formula (I) have a chiral center in the branched group off N-1. Thus, the compounds of Formula (I) can be resolved into compounds (or salts thereof) of Formulas (II) and (III) (or such compounds can be synthesized using well-known techniques using chiral starting materials):

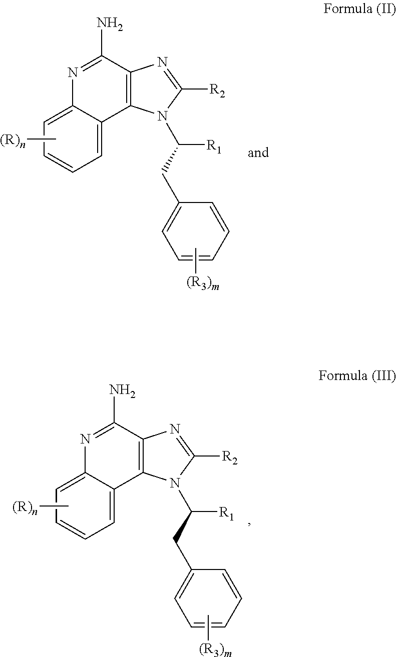

wherein n, R, R₁, R₂, and R₃ are as defined above.

The compounds and salts, such as pharmaceutically acceptable salts, of these compounds can be used as immune response modifiers due to their ability to induce cytokine biosynthesis (e.g., induce the synthesis of at least one cytokine) and otherwise modulate the immune response when administered to humans or animals. The compounds can therefore be used in the treatment of a variety of conditions such as viral diseases and tumors that are responsive to such changes in the immune response. The compounds can also be used as vaccine adjuvants when administered in combination with a vaccine.

Herein, when embodiments of Formulas (I), (II), and (III) are described, it is generally assumed that such statements refer to the compounds as well as the salts thereof.

Pharmaceutical compositions containing an effective amount of a compound (or salts thereof including pharmaceutically acceptable salts thereof) of Formula (I), such as a compound of Formula (II), Formula (III), or a combination thereof, are disclosed.

Also disclosed are methods of inducing cytokine biosynthesis in a human or animal, treating a viral disease in a human or animal, and treating a neoplastic disease in a human or animal by administering to the human or animal a compound of Formula (I), such as a compound of Formula (II), Formula (III), or a combination thereof, and/or pharmaceutically acceptable salt thereof.

The term “alkyl” refers to a monovalent group that is a radical of an alkane and includes straight-chain, branched, cyclic, and bicyclic alkyl groups, and combinations thereof Unless otherwise indicated, the alkyl groups typically contain from 1 to 20 carbon atoms. In some embodiments, the alkyl groups contain 1 to 10 carbon atoms, 1 to 6 carbon atoms, 1 to 4 carbon atoms, or 1 to 3 carbon atoms. Examples of “alkyl” groups include, but are not limited to, methyl, ethyl, n-propyl, n-butyl, n-pentyl, isobutyl, t-butyl, isopropyl, n-octyl, n-heptyl, ethylhexyl, cyclopentyl, cyclohexyl, cycloheptyl, adamantyl, norbornyl, and the like.

The term “alkoxy” refers to a monovalent group having an oxy group bonded directly to an alkyl group.

The term “C_{x-y}alkyl” and “C_{x-y}alkoxy” are inclusive of straight chain groups, branched chain groups, cyclic groups, and combinations thereof that have X to Y carbon atoms. For example, a “C₁₋₅alkyl” includes alkyl groups of 1 carbon, 2 carbons, 3 carbons, 4 carbons, and 5 carbons. Some examples of “C₁₋₅alkyl” include methyl, ethyl, n-propyl, isopropyl, n-butyl, sec-butyl, isobutyl, isomeric pentyls, cyclopropyl, cyclopentyl, and —CH₂-cyclopropyl.

The “salt” of a compound includes pharmaceutically acceptable salts, such as those described in Berge, Stephen M., “Pharmaceutical Salts,” Journal of Pharmaceutical Sciences, 1977, 66, pages 1-19. For example, salts can be prepared by reacting a free base compound (that is, one not in a salt form) with an inorganic or organic acid such as, for example, hydrochloric acid, sulfuric acid, hydrobromic acid, methane sulfonic acid, ethane sulfonic acid, malic acid, maleic acid, acetic acid, trifluoroacetic acid, para-toluenesulfonic acid, salicylic acid, succinic acid, tartaric acid, citric acid, pamoic acid, xinafoic acid, oxalic acid, and the like.

As used herein, “pharmaceutically acceptable carriers” include those carriers that can deliver therapeutically or prophylactically effective amounts of one or more of the compounds or salts of the disclosure to a subject by a chosen route of administration, are generally tolerated by the subject, and have an acceptable toxicity profile (preferably minimal to no toxicity at an administered dose). Some suitable pharmaceutically acceptable carriers are described in Remington's Pharmaceutical Sciences, 18^{th} Edition (1990), Mack Publishing Co. and can be readily selected by one of ordinary skill in the art. Typical pharmaceutically acceptable salts include hydrochloride and dihydrochloride.

“Effective amount” (including “therapeutically effective amount” and “prophylactically effective amount”) are defined as an amount of compound or salt sufficient to induce a therapeutic or prophylactic effect, such as cytokine induction, immunomodulation, antitumor activity, and/or antiviral activity. Depending on the disease or condition, the desired cytokine profile, and/or the acceptable level of side effects, the effective amount may vary. For example, a small amount of a very active compound or salt, or a large amount of a compound or salt of low activity, may be used to avoid undesirable side effects.

“Treat” and “treatment” as well as variations thereof refer to reducing, limiting progression, ameliorating, preventing, or resolving to any extent the symptoms or signs related to a condition.

“Ameliorate” and “ameliorating” refers to any reduction in the extent, severity, frequency, and/or likelihood of a symptom or clinical characteristic of a particular disease or condition.

“Antigen” refers to any substance that can be bound by an antibody in a manner that is immunospecific to some degree.

Herein, the term “comprises” and variations thereof do not have a limiting meaning where these terms appear in the description and claims. Such terms will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements. Any of the elements or combinations of elements that are recited in this specification in open-ended language (e.g., comprise and derivatives thereof), are considered to additionally be recited in closed-ended language (e.g., consist and derivatives thereof) and in partially closed-ended language (e.g., consist essentially, and derivatives thereof).

The words “preferred” and “preferably” refer to embodiments of the disclosure that may afford certain benefits, under certain circumstances. However, other claims may also be preferred, under the same or other circumstances. Furthermore, the recitation of one or more preferred claims does not imply that other claims are not useful, and is not intended to exclude other claims from the scope of the disclosure.

In this application, terms such as “a,” “an,” and “the” are not intended to refer to only a singular entity, but include the general class of which a specific example may be used for illustration. The terms “a,” “an,” and “the” are used interchangeably with the term “at least one.” The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise.

The term “and/or” means one or all of the listed elements or a combination of any two or more of the listed elements.

Also herein, all numbers are assumed to be modified by the term “about” and in certain embodiments, preferably, by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50).

Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

As used herein, the terms “ambient temperature” or “room temperature” refers to a temperature of 20° C. to 25° C. or 22° C. to 25° C.

The term “in the range” or “within a range” (and similar statements) includes the endpoints of the stated range.

Groupings of alternative elements or embodiments disclosed herein are not to be construed as limitations. Each group member may be referred to and claimed individually or in any combination with other members of the group or other elements found therein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

When a group is present more than once in a formula described herein, each group is “independently” selected, whether specifically stated or not. For example, when more than one R group is present in a formula, each R group is independently selected.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the invention. Thus, the appearances of such phrases in various places throughout this specification are not necessarily referring to the same embodiment of the invention. Furthermore, the particular features, configurations, compositions, or characteristics may be combined in any suitable manner in one or more embodiments.

The above summary of the present disclosure is not intended to describe each disclosed embodiment or every implementation of the present invention. The description that follows more particularly exemplifies illustrative embodiments. In several places throughout the application, guidance is provided through lists of examples, which examples may be used in various combinations. In each instance, the recited list serves only as a representative group and should not be interpreted as an exclusive list. Thus, the scope of the present disclosure should not be limited to the specific illustrative structures described herein, but rather extends at least to the structures described by the language of the claims, and the equivalents of those structures. Any of the elements that are positively recited in this specification as alternatives may be explicitly included in the claims or excluded from the claims, in any combination as desired. Although various theories and possible mechanisms may have been discussed herein, in no event should such discussions serve to limit the claimable subject matter.

### DETAILED DESCRIPTION OF ILLUSTRATIVE EMBODIMENTS

This disclosure provides compounds (or salts thereof) of the following Formula (I):

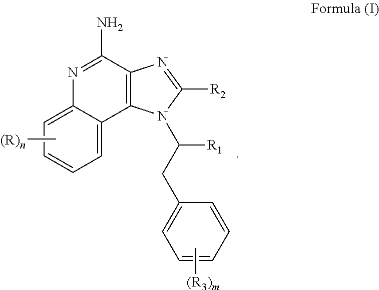

The compounds of Formula (I) have a chiral center in the branched group off N-1. Thus, the compounds of Formula (I) can be resolved into compounds (or salts thereof) of Formulas (II) and (III) (or such compounds can be synthesized using well-known techniques using chiral starting

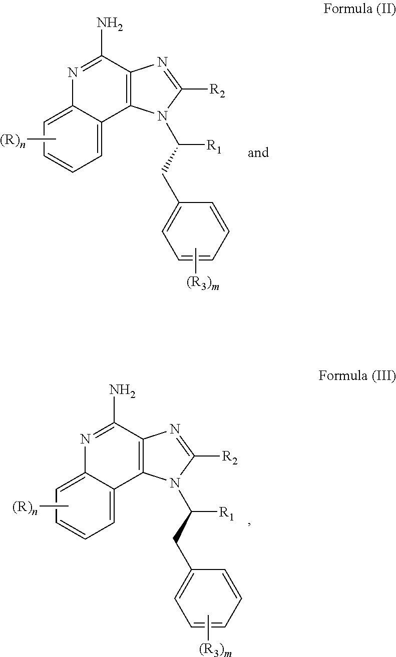

wherein:

m is an integer of 0 or 1;

n is an integer of 0 or 1;

R is selected from the group consisting of halogen, hydroxy, alkyl, alkoxy, and —C(O)—O-alkyl;

R₁ is alkyl or —CH₂—O—C₁₋₄alkyl;

R₂ is selected from the group consisting of hydrogen, methyl, ethyl, n-propyl, n-butyl, —CH₂OCH₃, —CH₂OCH₂CH₃, and —CH₂CH₂OCH₃; and

R₃ is selected from the group consisting of halogen, hydroxy, alkyl, and alkoxy; with the proviso that when R₃ is alkoxy, R₁ is alkyl.

Depending on the disease or condition, the desired cytokine profile, and/or the acceptable level of side effects, a compound or salt of Formula (II) may be more desirable than a compound or salt of Formula (III). Typically, compounds or salts of Formula (II) are more active with respect to inducing cytokine biosynthesis than compounds or salts of Formula (III). Whereas, generally a more active compound or salt of Formula (II) would be desirable for use, a less active compound or salt of Formula (III) may be used in certain situations, for example, to avoid undesirable side effects.

In Formulas (I), (II), and (III), R₁ is alkyl or —CH₂—O—C₁₋₄alkyl. In some embodiments of Formulas (I), (II), and (III), R₁ is —C₁₋₆alkyl or —CH₂—O—C₁₋₄alkyl. In some embodiments of Formulas (I), (II), and (III), R₁ is —C₁₋₆alkyl or —C₁₋₄alkyl. In some embodiments of Formulas (I), (II), and (III), R₁ is —CH₂—O—C₁₋₄alkyl, such as —CH₂—O—CH₃ or —CH₂—O—CH₂CH₃.

In Formulas (I), (II), and (III), R₂ is selected from the group consisting of hydrogen, methyl, ethyl, n-propyl, n-butyl, —CH₂OCH₃, —CH₂OCH₂CH₃, and —CH₂CH₂OCH₃. In some embodiments of Formulas (I), (II), and (III), R₂ is selected from the group consisting of hydrogen, methyl, and ethyl. In some embodiments of Formulas (I), (II), and (III), R₂ is hydrogen or methyl. In some embodiments of Formulas (I), (II), and (III), R₂ is hydrogen.

In Formulas (I), (II), and (III), n is 0 or 1 (i.e., R is present as a substituent on the aryl ring). In some embodiments of Formulas (I), (II), and (III), n is 0 (i.e., R is not present).

In some embodiments of Formulas (I), (II), and (III), n is 1, and R is selected from the group consisting of halogen, hydroxy, alkyl, alkoxy, and —C(O)—O-alkyl. In some embodiments of Formulas (I), (II), and (III), R is selected from the group consisting of halogen, hydroxy, —C₁₋₇alkyl, —C₁₋₇alkoxy, and —C(O)—O—C₁₋₅alkyl. In some embodiments, R is selected from the group consisting of hydroxy, F, and Cl. In some embodiments, R is selected from the group consisting of F and Cl.

In Formulas (I), (II), and (III), m is 0 or 1 (i.e., R₃ is present as a substituent on the aryl ring). In some embodiments of Formulas (I), (II), and (III), m is 0 (i.e., R₃ is not present).

In some embodiments of Formulas (I), (II), and (III), m is 1, and the —R₃ group is present and in an ortho, meta, or para position, whereas in some embodiments, the —R₃ group is in the para position. When present, R₃ is selected from the group consisting of halogen, hydroxy, alkyl, and alkoxy; with the proviso that when R₃ is alkoxy, R₁ is alkyl.

In some embodiments of Formulas (I), (II), and (III), R₃ is selected from the group consisting of halogen, hydroxy, —C₁₋₈alkyl, and —C₁₋₈alkoxy; with the proviso that when R₃ is —C₁₋₈alkoxy (i.e., —O—C₁₋₈alkyl), R₁ is alkyl (e.g., —C₁₋₆alkyl). In some embodiments of Formulas (I), (II), and (III), R₃ is —O—C₁₋₈alkyl, —O—C₁₋₆alkyl, or —O—C₁₋₄alkyl; with the proviso that when R₃ is one of these alkoxies, R₁ is an alkyl (e.g., a —C₁₋₆alkyl or a —C₁₋₄alkyl). In some embodiments of Formulas (I), (II), and (III), R₃ is selected from the group consisting of halogen, hydroxy, and —C₁₋₈alkyl. In some embodiments of Formulas (I), (II), and (III), R₃ is a —C₁₋₈alkyl, —C₁₋₆alkyl, or —C₁₋₄alkyl.

In some embodiments of Formulas (I), (II), and (III), m is 0; n is 0; R₁ is —C₁₋₆alkyl; and R₂ is selected from the group consisting of hydrogen, methyl, and ethyl. In some of these embodiments R₁ is —C₁₋₄alkyl and R₂ is hydrogen. Examples of such compounds include: 1-[(1R)-1-methyl-2-phenyl-ethyl]imidazo[4,5-c]quinolin-4-amine (Example 1); and 1-[(1R)-1-benzylpentyl]imidazo[4,5-c]quinolin-4-amine (Example 2).

In some embodiments of Formulas (I), (II), and (III), m is 0; n is 0; R₁ is —CH₂—O—C₁₋₄alkyl; and R₂ is selected from the group consisting of hydrogen, methyl, and ethyl. In some of these embodiments, R₂ is hydrogen. Examples of such compounds include:
1-[(1R)-1-benzyl-2-methoxy-ethyl]imidazo[4,5-c]quinolin-4-amine (Example 3);
1-[(1S)-1-benzyl-2-methoxy-ethyl]imidazo[4,5-c]quinolin-4-amine (Example 5);
1-[(1R)-1-benzyl-2-ethoxy-ethyl]imidazo[4,5-c]quinolin-4-amine (Example 4); and
1-[(1S)-1-benzyl-2-ethoxy-ethyl]imidazo[4,5-c]quinolin-4-amine (Example 6).

In some embodiments of Formulas (I), (II), and (III), m is 1; n is 0; R₁ is —C₁₋₆alkyl; R₂ is selected from the group consisting of hydrogen, methyl, and ethyl; and R₃ is —O—C₁₋₈alkyl. In some of these embodiments, R₁ is —C₁₋₆alkyl; R₂ is hydrogen; and R₃ is —O—C₁₋₆alkyl. Examples of such compounds include:
1-[(1R)-1-[(4-butoxyphenyl)methyl]pentyl]imidazo[4,5-c]quinolin-4-amine (Example 7).

In some embodiments of Formulas (I), (II), and (III), the compound is present in the form of a salt. The salt is typically a pharmaceutically acceptable salt. Most commonly the salt is a hydrochloride salt.

In some embodiments, mixtures of compounds of Formulas (II) and (III) are present. In some embodiments, the compound of Formula (II) has an enantiomeric purity of at least 80% enantiomeric excess (80% ee). The enantiomeric purity of a compound of Formula (II) is relative to a compound of Formula (III). In some embodiments, the compound of Formula (II) has an enantiomeric purity of at least 90% enantiomeric excess (90% ee). In some embodiments, the compound of Formula (II) has an enantiomeric purity of at least 95% enantiomeric excess (95% ee). In some embodiments, the compound of Formula (II) has an enantiomeric purity of at least 97% enantiomeric excess (97% ee). In some embodiments, the compound of Formula (II) has an enantiomeric purity of at least 98% enantiomeric excess (98% ee). In some embodiments, the compound of Formula (II) has an enantiomeric purity of at least 99% enantiomeric excess (99% ee). In some embodiments, the compound of Formula (II) has an enantiomeric purity of at least 99.5% enantiomeric excess (99.5% ee). In some embodiments, the compound of Formula (II) has an enantiomeric purity of at least 99.8% enantiomeric excess (99.8% ee).

Exemplary compounds of Formulas (I), (II), and (III) are presented in Tables 1-10. In the Tables 1-10, each row represents a specific compound with n, m, R₁, R₂, and R₃ defined.

TABLE 1

| n | m | R₁ | R₂ |
|---|---|---|---|
| 0 | 0 | —CH₃ | —H |
| 0 | 0 | —CH₂CH₃ | —H |
| 0 | 0 | —CH₂CH₂CH₃ | —H |
| 0 | 0 | —CH₂CH₂CH₂CH₃ | —H |
| 0 | 0 | —CH₂CH₂CH₂CH₂CH₃ | —H |
| 0 | 0 | —CH₂CH₂CH₂CH₂CH₂CH₃ | —H |
| 0 | 0 | —CH₂OCH₃ | —H |
| 0 | 0 | —CH₂OCH₂CH₃ | —H |
| 0 | 0 | —CH₂OCH₂CH₂CH₃ | —H |
| 0 | 0 | —CH₂OCH₂CH₂CH₂CH₃ | —H |

TABLE 2

| n | m | R₁ | R₂ | R₃ |
|---|---|---|---|---|
| 0 | 1 | —CH₃ | —H | —CH₃ |
| 0 | 1 | —CH₂CH₃ | —H | —CH₃ |
| 0 | 1 | —CH₂CH₂CH₃ | —H | —CH₃ |
| 0 | 1 | —CH₂CH₂CH₂CH₃ | —H | —CH₃ |
| 0 | 1 | —CH₂CH₂CH₂CH₂CH₃ | —H | —CH₃ |
| 0 | 1 | —CH₂CH₂CH₂CH₂CH₂CH₃ | —H | —CH₃ |
| 0 | 1 | —CH₂OCH₃ | —H | —CH₃ |
| 0 | 1 | —CH₂OCH₂CH₃ | —H | —CH₃ |
| 0 | 1 | —CH₂OCH₂CH₂CH₃ | —H | —CH₃ |
| 0 | 1 | —CH₂OCH₂CH₂CH₂CH₃ | —H | —CH₃ |

TABLE 3

| n | m | R₁ | R₂ | R₃ |
|---|---|---|---|---|
| 0 | 1 | —CH₃ | —H | —F |
| 0 | 1 | —CH₂CH₃ | —H | —F |
| 0 | 1 | —CH₂CH₂CH₃ | —H | —F |
| 0 | 1 | —CH₂CH₂CH₂CH₃ | —H | —F |
| 0 | 1 | —CH₂CH₂CH₂CH₂CH₃ | —H | —F |
| 0 | 1 | —CH₂CH₂CH₂CH₂CH₂CH₃ | —H | —F |
| 0 | 1 | —CH₂OCH₃ | —H | —F |
| 0 | 1 | —CH₂OCH₂CH₃ | —H | —F |
| 0 | 1 | —CH₂OCH₂CH₂CH₃ | —H | —F |
| 0 | 1 | —CH₂OCH₂CH₂CH₂CH₃ | —H | —F |

TABLE 4

| n | m | R₁ | R₂ | R₃ |
|---|---|---|---|---|
| 0 | 1 | —CH₃ | —H | —Cl |
| 0 | 1 | —CH₂CH₃ | —H | —Cl |
| 0 | 1 | —CH₂CH₂CH₃ | —H | —Cl |
| 0 | 1 | —CH₂CH₂CH₂CH₃ | —H | —Cl |
| 0 | 1 | —CH₂CH₂CH₂CH₂CH₃ | —H | —Cl |
| 0 | 1 | —CH₂CH₂CH₂CH₂CH₂CH₃ | —H | —Cl |
| 0 | 1 | —CH₂OCH₃ | —H | —Cl |
| 0 | 1 | —CH₂OCH₂CH₃ | —H | —Cl |
| 0 | 1 | —CH₂OCH₂CH₂CH₃ | —H | —Cl |
| 0 | 1 | —CH₂OCH₂CH₂CH₂CH₃ | —H | —Cl |

TABLE 5

| n | m | R₁ | R₂ | R₃ |
|---|---|---|---|---|
| 0 | 1 | —CH₃ | —H | —OH |
| 0 | 1 | —CH₂CH₃ | —H | —OH |
| 0 | 1 | —CH₂CH₂CH₃ | —H | —OH |
| 0 | 1 | —CH₂CH₂CH₂CH₃ | —H | —OH |
| 0 | 1 | —CH₂CH₂CH₂CH₂CH₃ | —H | —OH |
| 0 | 1 | —CH₂CH₂CH₂CH₂CH₂CH₃ | —H | —OH |
| 0 | 1 | —CH₂OCH₃ | —H | —OH |
| 0 | 1 | —CH₂OCH₂CH₃ | —H | —OH |
| 0 | 1 | —CH₂OCH₂CH₂CH₃ | —H | —OH |
| 0 | 1 | —CH₂OCH₂CH₂CH₂CH₃ | —H | —OH |

TABLE 6

| n | m | R₁ | R₂ | R₃ |
|---|---|---|---|---|
| 0 | 1 | —CH3 | —H | —OCH₃ |
| 0 | 1 | —CH₂CH₃ | —H | —OCH₃ |
| 0 | 1 | —CH₂CH₂CH₃ | —H | —OCH₃ |
| 0 | 1 | —CH₂CH₂CH₂CH₃ | —H | —OCH₃ |
| 0 | 1 | —CH₂CH₂CH₂CH₂CH₃ | —H | —OCH₃ |
| 0 | 1 | —CH₂CH₂CH₂CH₂CH₂CH₃ | —H | —OCH₃ |

TABLE 7

| n | m | R₁ | R₂ | R₃ |
|---|---|---|---|---|
| 0 | 1 | —CH₃ | —H | —OCH₂CH₃ |
| 0 | 1 | —CH₂CH₃ | —H | —OCH₂CH₃ |
| 0 | 1 | —CH₂CH₂CH₃ | —H | —OCH₂CH₃ |
| 0 | 1 | —CH₂CH₂CH₂CH₃ | —H | —OCH₂CH₃ |
| 0 | 1 | —CH₂CH₂CH₂CH₂CH₃ | —H | —OCH₂CH₃ |
| 0 | 1 | —CH₂CH₂CH₂CH₂CH₂CH₃ | —H | —OCH₂CH₃ |

TABLE 8

| n | m | R₁ | R₂ | R₃ |
|---|---|---|---|---|
| 0 | 1 | —CH₃ | —H | —OCH₂CH₂CH₂CH₃ |
| 0 | 1 | —CH₂CH₃ | —H | —OCH₂CH₂CH₂CH₃ |
| 0 | 1 | —CH₂CH₂CH₃ | —H | —OCH₂CH₂CH₂CH₃ |
| 0 | 1 | —CH₂CH₂CH₂CH₃ | —H | —OCH₂CH₂CH₂CH₃ |
| 0 | 1 | —CH₂CH₂CH₂CH₂CH₃ | —H | —OCH₂CH₂CH₂CH₃ |
| 0 | 1 | —CH₂CH₂CH₂CH₂CH₂CH₃ | —H | —OCH₂CH₂CH₂CH₃ |

TABLE 9

| n | m | R₁ | R₂ | R₃ |
|---|---|---|---|---|
| 0 | 1 | —CH₃ | —H | —OC₆H₁₃ |
| 0 | 1 | —CH₂CH₃ | —H | —OC₆H₁₃ |
| 0 | 1 | —CH₂CH₂CH₃ | —H | —OC₆H₁₃ |
| 0 | 1 | —CH₂CH₂CH₂CH₃ | —H | —OC₆H₁₃ |
| 0 | 1 | —CH₂CH₂CH₂CH₂CH₃ | —H | —OC₆H₁₃ |
| 0 | 1 | —CH₂CH₂CH₂CH₂CH₂CH₃ | —H | —OC₆H₁₃ |

TABLE 10

| n | m | R₁ | R₂ | R₃ |
|---|---|---|---|---|
| 0 | 1 | —CH₃ | —H | —OC₈H₁₇ |
| 0 | 1 | —CH₂CH₃ | —H | —OC₈H₁₇ |
| 0 | 1 | —CH₂CH₂CH₃ | —H | —OC₈H₁₇ |
| 0 | 1 | —CH₂CH₂CH₂CH₃ | —H | —OC₈H₁₇ |
| 0 | 1 | —CH₂CH₂CH₂CH₂CH₃ | —H | —OC₈H₁₇ |
| 0 | 1 | —CH₂CH2CH₂CH₂CH₂CH₃ | —H | —OC₈H₁₇ |

The disclosure provides a method of inducing cytokine biosynthesis in a human or animal by administering to the human or animal an effective amount of a compound or salt selected from the group consisting of any one of the above embodiments of Formula (I), which may be compounds of Formula (II) and/or Formula (III) (preferably, Formula (II)), or salts thereof.

The disclosure provides a method of inducing IFN-alpha biosynthesis in a human or animal by administering to the human or animal an effective amount of a compound or salt selected from any one of the above embodiments of Formula (I), which may be compounds of Formula (II) and/or Formula (III) (preferably, Formula (II)), or salts thereof.

The disclosure provides a method of inducing IFN-gamma biosynthesis in a human or animal by administering to the human or animal an effective amount of a compound or salt selected from any one of the above embodiments of Formula (I), which may be compounds of Formula (II) and/or Formula (III) (preferably, Formula (II)), or salts thereof.

The disclosure provides a method of inducing TNF-alpha biosynthesis in a human or animal by administering to the human or animal an effective amount of a compound or salt selected from any one of the above embodiments of Formula (I), which may be compounds of Formula (II) and/or Formula (III) (preferably, Formula (II)), or salts thereof.

The disclosure provides a method of inducing IP-10 biosynthesis in a human or animal by administering to the human or animal an effective amount of a compound or salt selected from any one of the above embodiments of Formula (I), which may be compounds of Formula (II) and/or Formula (III) (preferably, Formula (II)), or salts thereof.

The disclosure provides a method for treating a viral disease in a human or animal by administering to the human or animal an effective amount of a compound or salt selected from any one of the above embodiments of Formula (I), which may be compounds of Formula (II) and/or Formula (III) (preferably, Formula (II)), or salts thereof.

The disclosure provides a method for treating a neoplastic disease in a human or animal by administering to the human or animal an effective amount of a compound or salt selected from any one of the above embodiments of Formula (I), which may be compounds of Formula (II) and/or Formula (III) (preferably, Formula (II)), or salts thereof.

Compounds of the disclosure may be synthesized by synthetic routes that include processes analogous to those well known in the chemical arts, particularly in light of the description contained herein. The starting materials are generally available from commercial sources such as the Sigma-Aldrich Company (St. Louis, Mo.) or are readily prepared using methods well known to those of ordinary skill in the art (e.g., prepared by methods generally described in Louis F. Fieser and Mary Fieser, Reagents for Organic Synthesis, v. 1-26, Wiley, New York; Alan R. Katritsky, Otto Meth-Cohn, Charles W. Rees, Comprehensive Organic Functional Group Transformations, v 1-6, Pergamon Press, Oxford, England, (1995); Barry M. Trost and Ian Fleming, Comprehensive Organic Synthesis, v. 1-8, Pergamon Press, Oxford, England, (1991); or Beilsteins Handbuch der Organischen Chemie, 4, Aufl. Ed. Springer-Verlag, Berlin, Germany, including supplements (also available via the Beilstein online database)).

Compounds of the disclosure can be prepared, for example, according to Reaction Schemes I, II, III, IV, and V where R, R₁, R₂, R₃, m, and n are as described above. In Reaction Scheme I, a 4-chloro-3-nitroquinoline of Formula V is reacted in step (1) with an amine compound of Formula IV to provide a 3-nitroquinolin-4-amine of Formula VI. The reaction can be carried out by adding the amine of Formula IV to a solution of Formula V in a suitable solvent such as dichloromethane in the presence of a tertiary amine such as triethylamine. The 4-chloro-3-nitroquinoline compound of Formula V and substituted analogs are known compounds (see, for example, U.S. Pat. No. 3,700,674 (Diehl et al.), U.S. Pat. No. 5,389,640 (Gerster et al.), U.S. Pat. No. 6,110,929 (Gerster et al.), U.S. Pat. No. 7,923,560 (Wightman et al.), and references cited therein). In many cases, substituted analogs of Formula V (for example n=1 and R being a halogen, alkoxy or benzyloxy group) can be prepared starting with commercially available substituted anilines.

In step (2) of Reaction Scheme I, the nitro group of Formula VI can be reduced to an amino group. The reduction can be carried out in a pressure bottle using hydrogen, a catalytic amount of palladium or platinum on carbon, and a solvent such as methanol, acetonitrile, toluene, or combinations thereof The reaction can be carried out with a Parr apparatus. Alternatively, the desired reduction can be accomplished using sodium dithionite and catalytic dioctyl viologen in a two phase dichloromethane-water solvent system. In step (3) of Reaction Scheme I, the resulting 3,4-diamine compound can be reacted with a carboxylic acid (R₂CO₂H) to provide a 1H-imidazo[4,5-c]quinoline of Formula VII. Suitable equivalents to carboxylic acids such as acyl chlorides, thioesters, and 1,1-dialkoxyalkyl alkanoates can also be used. The carboxylic acid or equivalent is selected so that it will provide the desired R₂ substituent in a compound of Formula VII. For example, triethylorthoformate will provide a compound where R₂ is hydrogen and trimethyl orthovalerate will provide a compound where R₂ is n-butyl. The reaction can be carried out without a solvent or with an inert solvent (for example ethyl acetate, n-propyl acetate or toluene). Optionally, a catalyst such as pyridine hydrochloride can be included.

In step (4) of Reaction Scheme I, the 1H-imidazo[4,5-c]quinoline of Formula VII can be oxidized to provide a 1H-imidazo[4,5-c]quinoline-5N-oxide using a conventional oxidizing agent capable of forming an N-oxide. Preferably, a solution of the compound of Formula VII in a suitable solvent such as chloroform or dichloromethane is reacted with 3-chloroperbenzoic acid (MCPBA) at ambient temperature.

In step (5) of Reaction Scheme I, the N-oxide compound can be aminated to provide a 1H-imidazo[4,5-c]quinoline-4-amine of Formula I. Step (5) involves reacting the N-oxide compound with an acylating agent and an aminating agent in an inert solvent such as dichloromethane or chloroform. Suitable acylating agents include alkyl- or arylsulfonyl chlorides such as benzenesulfonyl chloride, methanesulfonyl chloride, or para-toluenesulfonyl chloride. Ammonium hydroxide is a suitable aminating agent. The compound of Formula I can optionally be isolated as an organic or inorganic salt (for example as an HCl salt).

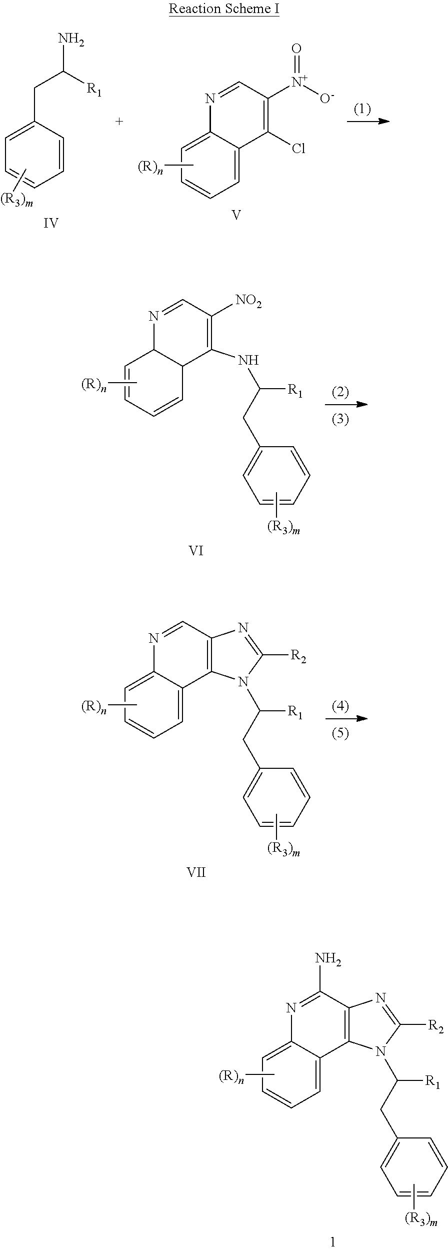

In Reaction Scheme II, the compound of Formula VIII is a Boc-protected alpha-amino acid. Many Boc-protected alpha-amino acids are commercially available (for example N-Boc phenylalanine, N-Boc-2-fluorophenylalanine, N-Boc-3-fluorophenylalanine, N-Boc-4-fluorophenylalanine, N-Boc-4-chlorophenylalanine, N-Boc-4-bromophenylalanine, N-Boc-4-iodophenylalanine, N-Boc tyrosine and N-Boc-O-tert-butyl-L-tyrosine). Boc-protected alpha-amino acids can also be prepared from alpha-amino acids by a number of conventional methods (for example, see P. G. M. Wuts, Greene's Protective Groups in Organic Synthesis, John Wiley & Sons, New York, USA, 2014). In step (6) of Reaction Scheme II, a Boc protected alpha-amino acid of Formula VIII can be dissolved in an inert solvent such as tetrahydrofuran and reacted with an alkyl chloroformate (for example methyl chloroformate, ethyl chloroformate or iso-butyl chloroformate) in the presence of a base (for example triethylamine or N-methylmorpholine) to form a mixed anhydride. The intermediate mixed anhydride can then be reduced with sodium borohydride to provide an alcohol of Formula IX. Many Boc protected amino alcohols of Formula IX are commercially available (for example example tert-butyl N-[(1R)-1-benzyl-2-hydroxy-ethyl]carbamate and tert-butyl N-[(1S)-1-benzyl-2-hydroxy-ethyl]carbamate) eliminating the need for step (6).

In step (7) of Reaction Scheme II, the alcohol of Formula IX can be converted to an iodide using conventional methods such as adding the alcohol to a mixture of triphenylphosphine, imidazole and iodine in an inert solvent (for example methylene chloride) to provide the alkyl iodide of formula X. In step (8) of Reaction Scheme II, the iodide can be reduced to provide a compound of Formula XI. The reduction can be carried out in a pressure bottle using hydrogen, a catalytic amount of palladium or platinum on carbon, and a solvent such as methanol. The reduction can be performed in the presence of a base such as sodium bicarbonate. The reaction can be carried out with a Parr apparatus. In step (9), the Boc amino protecting group in can be removed by reacting with hydrochloric acid in an alcohol solvent (for example methanol or ethanol) to provide the primary amine compound of Formula XII. It is often convenient to isolate the compound of Formula XII as a hydrochloride salt. The compound of Formula XII can be further reacted according to steps (1-5) described in Reaction Scheme Ito provide compounds of Formula I where R₁ is —CH₃.

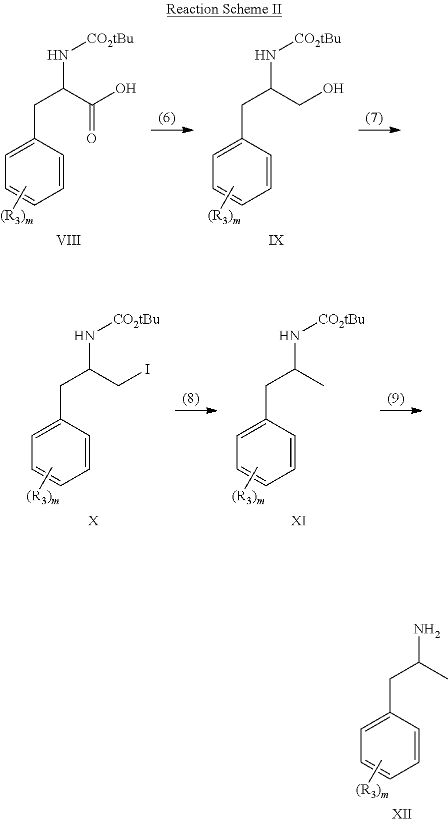

In step (10) of Reaction Scheme III, an alpha-amino alcohol of Formula IX can be oxidized to an aldehyde by a variety of methods known to one skilled in the art. In particular, the method described by D. A. Six et al. (J. Med. Chem., 2007, 50, pages 4222-4235) using (2,2,6,6-tetramethylpiperidin-1-yl)oxyl (TEMPO) and sodium hypochlorite can be employed to oxidize Boc protected amino alcohols of Formula IX to aldehydes of Formula XIII.

In step (11) of Reaction Scheme III, the aldehyde of Formula XIII can be subjected to Wittig reaction conditions to provide the olefin compound of Formula XIV (where R₄ is —H or C₁₋₄alkyl). In the Wittig reaction, alkyl triphenylphosphonium salts can be reacted with a base to form a phosphorus-carbon ylide. Examples of suitable alkyl triphenylphosphonium salts include methyl triphenylphosphonium bromide, ethyl triphenylphosphonium bromide, n-propyl triphenylphosphonium bromide and the like. Examples of suitable bases include sodium hydride, butyl lithium and potassium hexamethyldisilazide. The aldehyde of Formula XIII can then be reacted with the triphenylphosphonium ylide in a suitable solvent such as toluene to provide the olefin compound of Formula XIV. The obtained olefin is typically formed in the Z-configuration (as drawn), but in some instances can also be in the E-configuration.

In step (12) of Reaction Scheme III, the olefin of Formula XIV can be reduced to form a saturated alkyl group of Formula XV. The reduction can be carried out in a pressure bottle using hydrogen, a catalytic amount of palladium or platinum on carbon, and a solvent such as methanol, acetonitrile, toluene, or combinations thereof The reaction can be carried out with a Parr apparatus. In step (13), the Boc amino protecting group can be removed by reacting with hydrochloric acid in an alcohol solvent (for example methanol or ethanol) to provide the primary amine compound of Formula XVI. It is often convenient to isolate the compound of Formula XVI as a hydrochloride salt. The compound of Formula XVI can be further reacted according to steps (1-5) described in Reaction Scheme Ito provide compounds of Formula I where R₁ is an alkyl group having at least two carbon atoms.

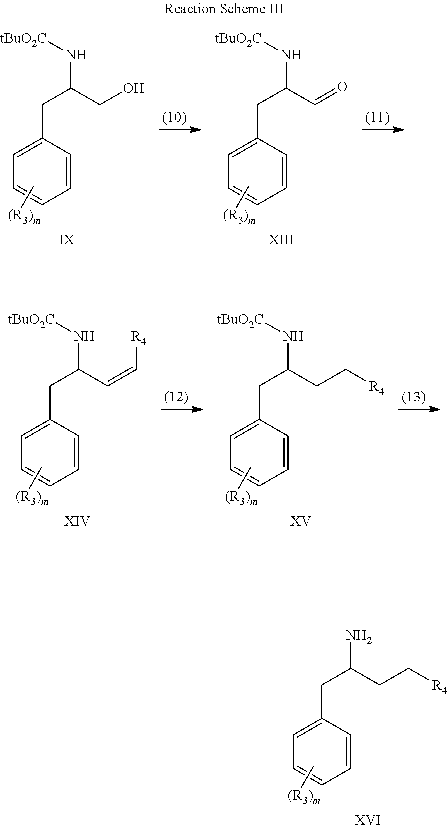

In step (14) of Reaction Scheme IV, the compound of Formula IX can be alkylated to give an alkyl ether of Formula XVII. The compound of Formula IX dissolved in an inert solvent (such as heptane or toluene) can be reacted with a dialkyl sulfate (such as for example dimethyl sulfate or diethyl sulfate) in the presence of sodium hydroxide and a phase transfer catalyst (such as tetrabutylammonium bromide) to provide the alkyl ether of Formula XVII. The Boc amino protecting group can be removed in step (15) by reacting the compound of Formula XVII with hydrochloric acid in an alcohol solvent (for example methanol or ethanol) to provide the primary amine compound of Formula XVIII. It is often convenient to isolate the compound of Formula XVIII as a hydrochloride salt. The compound of Formula XVIII can be further reacted according to steps (1-5) described in Reaction Scheme Ito provide compounds of Formula I where R₁ is —CH₂—O—C₁₋₄alkyl.

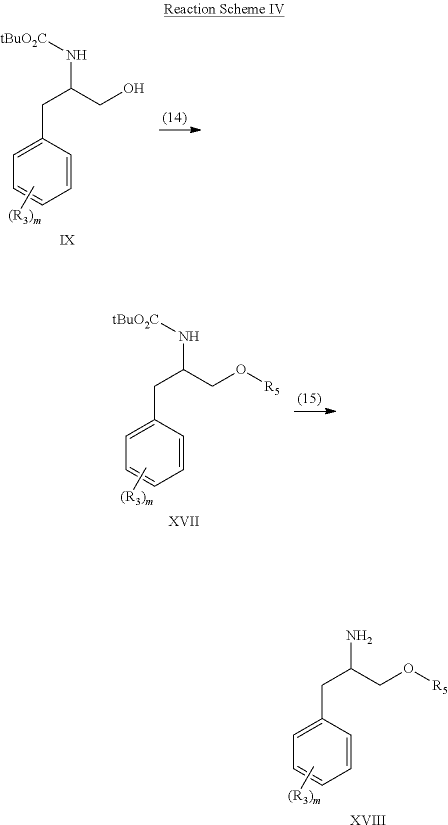

In Reaction Scheme V, the compound of Formula XIX is a protected phenol where R₆ is a suitable protecting group for a phenolic alcohol (such as a tert-butyl or a benzyl protecting group). The compound of Formula XIX can be prepared using methods described in Reaction Scheme II to provide compounds where R₁ is —CH₃ or using methods described in Reaction Scheme III to provide compounds where R₁ is an alkyl group having at least two carbon atoms. In step (16), the Boc amino protecting group and the phenolic alcohol protecting group can both be removed by reacting with hydrochloric acid in an alcohol solvent (for example methanol or ethanol) to provide the primary amine compound of Formula XX. It is often convenient to isolate the compound of Formula XX as a hydrochloride salt.

In step (17) of Reaction Scheme V, the compound of Formula XX can be reacted with a 4-chloro-3-nitroquinoline of Formula V to provide a 3-nitroquinolin-4-amine of Formula XXI. The reaction can be carried out by adding the amine of Formula XX to a solution of Formula V in a suitable solvent such as dichloromethane in the presence of a tertiary amine such as triethylamine.

In step (18) of Reaction Scheme V, the nitro group of Formula XXI can be reduced to an amino group. The reduction can be carried out in a pressure bottle using hydrogen, a catalytic amount of palladium or platinum on carbon, and a solvent such as methanol, acetonitrile, toluene, or combinations thereof The reaction can be carried out with a Parr apparatus. Alternatively, the desired reduction can be accomplished using sodium dithionite and catalytic dioctyl viologen in a two phase dichloromethane-water solvent system. In step (19) of Reaction Scheme V, the resulting 3,4-diamine compound can be reacted with a carboxylic acid (R₂CO₂H) to provide a 1H-imidazo[4,5-c]quinoline of Formula XXII. Suitable equivalents to carboxylic acids such as acyl chlorides, thioesters, and 1,1-dialkoxyalkyl alkanoates can also be used. The carboxylic acid or equivalent is selected so that it will provide the desired R₂ substituent in a compound of Formula XXII. For example, triethylorthoformate will provide a compound where R₂ is hydrogen and trimethyl orthovalerate will provide a compound where R₂ is n-butyl. The reaction can be carried out without a solvent or with an inert solvent (for example ethyl acetate, n-propyl acetate or toluene). Optionally, a catalyst such as pyridine hydrochloride can be included.

In step (20) of Reaction Scheme V, the 1H-imidazo[4,5-c]quinoline of Formula XXII is converted to an ether of Formula XXIII using conventional synthetic methods. For example, the compound of Formula XXII can be reacted with a suitable alkyl halide (alkyl bromide or alkyl iodide) and a base (such as cesium carbonate) in an inert solvent (such as N,N-dimethylformamide). The alkyl halide is selected so that it will provide the desired R₇ substituent in the compound of Formula XXIII.

In step (21) of Reaction Scheme V, the 1H-imidazo[4,5-c]quinoline of Formula XXIII can be oxidized to provide a 1H-imidazo[4,5-c]quinoline-5N-oxide using a conventional oxidizing agent capable of forming an N-oxide. Preferably, a solution of the compound of Formula XXIII in a suitable solvent such as chloroform or dichloromethane is reacted with 3-chloroperbenzoic acid (MCPBA) at ambient temperature.

In step (22) of Reaction Scheme V, the N-oxide compound can be aminated to provide a 1H-imidazo[4,5-c]quinoline-4-amine of Formula XXIV. Step (22) involves reacting the N-oxide compound with an acylating agent and an aminating agent in an inert solvent such as dichloromethane or chloroform. Suitable acylating agents include alkyl- or arylsulfonyl chlorides such as benzenesulfonyl chloride, methanesulfonyl chloride, or para-toluenesulfonyl chloride. Ammonium hydroxide is a suitable aminating agent. The compound of Formula XXIV can optionally be isolated as an organic or inorganic salt (for example as an HCl salt). Formula XXIV is an embodiment of Formula I where R₁ is alkyl and R₃ is alkoxy.

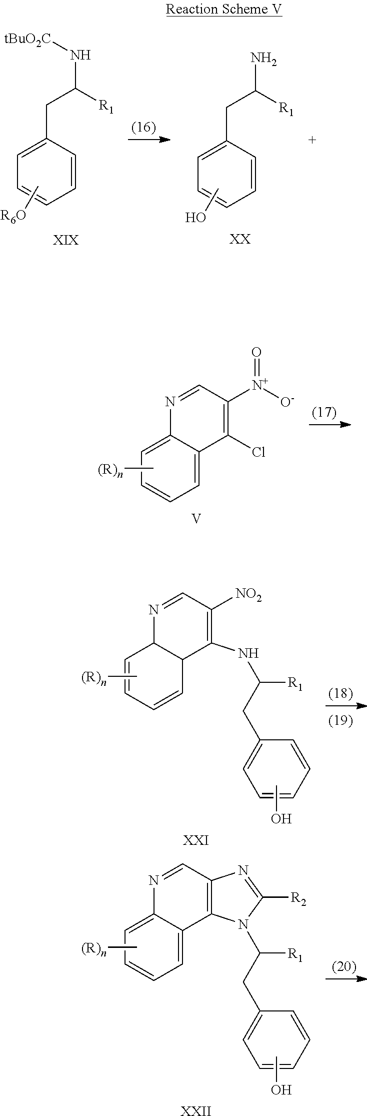

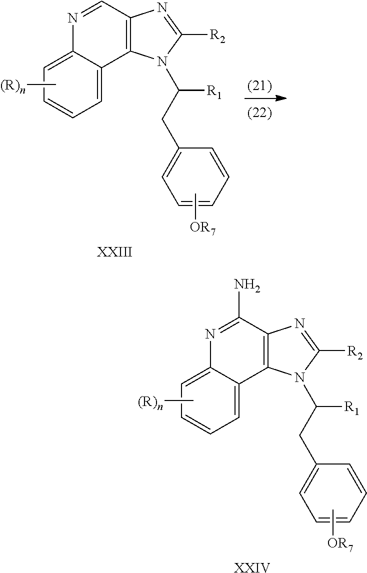

For Reaction Schemes I-V, the compounds are drawn as racemic. It is understood that these reaction schemes can also be followed starting with compounds of high enantiomeric purity (for example a D or L amino acids) to prepare final compounds of the disclosure in high enantiomeric purity.

Compounds of Formula (I), which may be compounds of Formula (II) and/or Formula (III), can be prepared by starting the reaction scheme with reactants having high enantiomeric purity. Alternatively, a racemic mixture of reactants or reactants of low enantiomeric purity (for example 10-70% enantiomeric excess) can be used with the final product isolated as the desired Formula (II) enantiomer using any suitable procedure for the resolution of a mixture of enantiomers. A well-known method for the resolution of a mixture of enantiomers is HPLC chromatography using a column with a chiral stationary phase (CSP). Another standard method for the resolution of a mixture of enantiomers involves reacting the mixture with an optically pure carboxylic acid to form diastereomeric salts that can be readily separated by for example recrystallization or chromatography methods. Regeneration of the free base completes the resolution process. Examples of resolving agents that are available in high enantiomeric purity include, but are not limited to, (+)-tartaric acid, (−)-mandelic acid, (−)-malic acid, (+)-camphor-10-sulfonic acid, and (+)-2,3-dibenzoyltartaric acid. If needed, different types of resolution steps can be combined and multiple resolution steps can be utilized to achieve the desired enantiomeric purity. The enantiomeric purity is represented as the percent enantiomeric excess (% ee). Methods for the resolution of isomers are described in the references: Y. Okamoto, Chemical Society Reviews, 2008, 37, pages 2593-2608; G. Gubitz, Biopharmaceutics and Drug Disposition, 2001, 22, pages 291-336; and S. Mane, Analytical Methods, 2016, 8, pages 7567-7586.

In the preparation of the compounds of the disclosure it is understood by one of ordinary skill in the art that it may be necessary to protect a particular functional group while reacting other functional groups of an intermediate compound. The need for such protection will vary depending on the nature of the particular functional group and the conditions of the particular reaction step. A review of reactions for protecting and deprotecting functional groups can be found in P. G. M. Wuts, Greene's Protective Groups in Organic Synthesis, John Wiley & Sons, New York, USA, 2014.

Conventional methods and techniques of separation and purification can be used to isolate the IRM compounds used in the compositions of the disclosure. Such techniques may include, for example, all types of chromatography (high performance liquid chromatography (HPLC), column chromatography using common absorbents such as silica gel, and thin layer chromatography), recrystallization, and differential (i.e., liquid-liquid) extraction techniques.

The enantiomeric excess of the compounds of the disclosure can be determined using standard analytical assays such as gas chromatography or HPLC with a column having a chiral stationary phase (CSP). Suitable columns with a CSP are available from Chiral Technologies, Inc., Westchester, Pa.

Enantiomeric excess (% ee) is calculated according to Equation 1.

$$enantiomeric excess ( \% ee ) = \frac{( mole \% of major enantiomer ) - ( mol \% of minor enatiomer )}{( mole \% of major enantiomer ) + ( mole \% of minor enantiomer )} \times 100. \qquad Equation 1$$

Enantiomeric excess (% ee) can be calculated from a chiral HPLC chromatogram by comparing the peak areas of the major enantiomer and minor enantiomer signals according to Equation 2.

$$enantiomeric excess ( \% ee ) = \frac{( peak area of major enantiomer ) - ( peak area of minor enantiomer )}{( peak area of major enantiomer ) + ( peak area of minor enantiomer )} \times 100. \qquad Equation 2$$

Prodrugs of the disclosed compounds can also be prepared by attaching to the compounds a functional group that can be cleaved under physiological conditions. Typically, a cleavable functional group will be cleaved in vivo by various mechanisms (such a through a chemical (e.g., hydrolysis) or enzymatic transformation) to yield a compound of the disclosure. A discussion of the use of prodrugs is provided by T. Higuchi and W. Stella. “Prodrugs as Novel Delivery Systems”, vol. 14 of the ACS Symposium Series, and in Bioreversible Carriers in Drug Design, ed. Edward B. Roche, American Pharmaceutical Association and Pergamon Press, 1987.

### Pharmaceutical Compositions and Biological Activity

Pharmaceutical compositions of the disclosure are also contemplated. Pharmaceutical compositions of the disclosure contain a therapeutically effective amount of a compound or salt of the disclosure (described herein) in combination with a pharmaceutically acceptable carrier.

The compounds of Formula (I), which may be compounds of Formula (II) and/or Formula (III), may be provided in any pharmaceutical composition suitable for administration to a subject (human or animal) and may be present in the pharmaceutical composition in any suitable form (for example as a solution, a suspension, an emulsion, or any form of a mixture). The pharmaceutical composition may be formulated with any pharmaceutically acceptable excipient, carrier, or vehicle. In some embodiments, the pharmaceutically acceptable carrier comprises water (for example phosphate buffered saline or citrate buffered saline). In some embodiments, the pharmaceutically carrier comprises an oil (for example corn, sesame, cottonseed, soybean, or safflower oil). The pharmaceutical composition may further include one or more additives including suspending agents, surfactants, dispersing agents, and preservatives (such as an anti-oxidant).

In some embodiments of the pharmaceutical composition, the compounds of Formula (I), which may be compounds of Formula (II) and/or Formula (III), can be incorporated in a homogeneously dispersed formulation. In some embodiments of the pharmaceutical composition, the compounds of Formula (I), which may be compounds of Formula (II) and/or Formula (III), can be incorporated in an emulsified formulation. In some embodiments of the pharmaceutical composition, the compounds of Formula (I), which may be compounds of Formula (II) and/or Formula (III), can be incorporated in an oil-in-water formulation. An oil-in-water formulation can comprise an oil component, an aqueous component, and one or more surfactants (for example formulations comprising soybean oil, TWEEN 80, SPAN 85, and phosphate buffered saline). In some embodiments of the pharmaceutical composition, the compounds of Formula (I), which may be compounds of Formula (II) and/or Formula (III), can be incorporated into a liposome formulation.

In some embodiments, the pharmaceutical composition can further comprise an antigen in an amount effective to generate an immune response against the antigen. In some embodiments, the antigen is a vaccine.

The pharmaceutical composition can be administered in any suitable manner (parenterally or non-parenterally). In some embodiments, the pharmaceutical composition can be administered by an intradermal, subcutaneous, intramuscular, or intravenous injection.

In any embodiment of a pharmaceutical composition comprising a compound of Formula (II), the compound of Formula (II) is present in the composition in at least 80% enantiomeric excess, relative to the compound of Formula (III), at least 90% enantiomeric excess, at least 95% enantiomeric excess, at least 96% enantiomeric excess, at least 96% enantiomeric excess, at least 97% enantiomeric excess, at least 98% enantiomeric excess, at least 99% enantiomeric excess, at least 99.5% enantiomeric, or at least 99.8% enantiomeric excess.

In any embodiment of a pharmaceutical composition comprising a compound of Formula (II), the opposite enantiomer to the compound of Formula (II) (i.e., compound of Formula (III) is present in the composition in less than 10%, less than 5%, less than 2.5%, less than 2%, less than 1.5%, less than 1%, less than 0.5%, less than 0.25%, or less than 0.1%.

The exact amount of compound or salt used in a pharmaceutical composition of the disclosure will vary according to factors known to those of skill in the art, such as the physical and chemical nature of the compound or salt, the nature of the carrier, and the intended dosing regimen.

In some embodiments, the concentration of a compound of Formula (I), which may be a compound of Formula (II) and/or Formula (III), in the pharmaceutical composition can be at least 0.0005 mg/mL, at least 0.001 mg/mL, or at least 0.05 mg/mL. In some embodiments, the concentration of a compound of Formula (I), which may be a compound of Formula (II) and/or Formula (III), in the pharmaceutical composition can be up to 2.4 mg/mL, up to 0.06 mg/mL, up to 0.01 mg/mL, or up to 0.005 mg/mL.

In some embodiments, the compositions of the disclosure will contain sufficient active ingredient or prodrug to provide a dose of at least 100 nanograms per kilogram (ng/kg), or at least 10 micrograms per kilogram (μg/kg), of the compound or salt to the subject. In some embodiments, the compositions of the disclosure will contain sufficient active ingredient or prodrug to provide a dose of up to 50 milligrams per kilogram (mg/kg), or up to 5 mg/kg, of the compound or salt to the subject.

In some embodiments, the compositions of the disclosure will contain sufficient active ingredient or prodrug to provide a dose of, for example, from 0.01 mg/m² to 5.0 mg/m², computed according to the Dubois method, in which the body surface area of a subject (m²) is computed using the subject's body weight: m²=(wt kg^{0.425}×height cm^{0.725})×0.007184, although in some embodiments the methods may be performed by administering a compound or salt or composition in a dose outside this range. In some of these embodiments, the method includes administering sufficient compound to provide a dose of from 0.1 mg/m² to 2.0 mg/m² to the subject, for example, a dose of from 0.4 mg/m² to 1.2 mg/m².

A variety of dosage forms may be used to administer the compounds or salts of the disclosure to a human or animal. Dosage forms that can be used include, for example, tablets, lozenges, capsules, parenteral formulations, creams, ointments, topical gels, aerosol formulations, liquid formulations (e.g., aqueous formulation), transdermal patches, and the like. These dosage forms can be prepared with conventional pharmaceutically acceptable carriers and additives using conventional methods, which generally include the step of bringing the active ingredient into association with the carrier. A preferred dosage form has one or more of compounds or salts of the disclosure dissolved in an aqueous formulation.

Compounds or salts disclosed herein induce the production of certain cytokines in experiments performed according to the description of the Examples. These results indicate that the compounds or salts are useful for enhancing the immune response in a number of different ways, making them useful in the treatment of a variety of disorders.

The compounds or salts described herein can be administered as the single therapeutic agent in the treatment regimen, or the compounds or salts described herein may be administered in combination with other active agents, including antivirals, antibiotics, proteins, peptides, oligonucleotides, antibodies, etc.

Compounds or salts described herein induce the production of cytokines (e.g., IFN-alpha, IFN-gamma, TNF-alpha, IP-10) in experiments performed according to the tests set forth below. These results indicate that the compounds of the disclosure or salts are useful for activating the immune response in a number of different ways, rendering them useful in the treatment of a variety of disorders. As such, the compounds or salts of the disclosure (particularly compounds or salts of Formula II) are agonists of cytokine biosynthesis and production, particularly agonists of IFN-alpha, IFN-gamma, TNF-alpha, and IP-10 cytokine biosynthesis and production.

It is believed that one way in which the compounds or salts of the disclosure (particularly compounds or salts of Formula II) induce cytokine production is through the activation of Toll-like receptors (TLRs) in the immune system, particularly TLR-7 and/or TLR-8, however other mechanisms may be involved. It is believed that in the immune system pathways (i.e., mechanisms) for cytokine induction, the compounds or salts of the disclosure (particularly compounds or salts of Formula II) primarily act as agonists of TLR-7 and/or TLR-8, however, other pathways or activities may be involved.

Administration of the compounds (particularly one of Formula (II)) or salts described herein can induce the production of interferon-alpha (IFN-alpha), interferon-gamma (IFN-gamma), tumor necrosis factor-alpha (TNF-alpha), and IP-10 in cells. Cytokines whose biosynthesis can be induced by compounds or salts of the disclosure include IFN-alpha, IFN-gamma, TNF-alpha, IP-10, and a variety of other cytokines. Among other effects, these cytokines can inhibit virus production and tumor cell growth, making the compounds or salts useful in the treatment of viral diseases and neoplastic diseases. Accordingly, the disclosure provides a method of inducing cytokine biosynthesis in a human or animal by administering an effective amount of a compound or salt of the disclosure to the human or animal. The human or animal to which the compound or salt is administered for induction of cytokine production may have one or more diseases, disorders, or conditions described below, for example a viral disease or a neoplastic disease, and administration of the compound or salt may provide therapeutic treatment. Alternatively, the compound or salt may be administered to the human or animal prior to the human or animal acquiring the disease so that administration of the compound or salt may provide a prophylactic treatment.

In addition to the ability to induce the production of cytokines, compounds (particularly one of Formula (II)) or salts thereof described herein can affect other aspects of the innate immune response. For example, natural killer cell activity may be stimulated, an effect that may be due to cytokine induction. The compounds described herein (particularly one of Formula (II)) or salts thereof may also activate macrophages, which in turn stimulate secretion of nitric oxide and the production of additional cytokines. In addition, the compounds described herein (particularly one of Formula (II)) or salts thereof may cause proliferation and differentiation of B-lymphocytes.

Conditions for which compounds (particularly one of Formula (II)) or salts thereof or compositions identified herein may be used as treatment include, but are not limited to:

Viral diseases such as, for example, diseases resulting from infection by an adenovirus, a herpes virus (e.g., HSV-I, HSV-II, CMV, or VZV), a poxvirus (e.g., an orthopoxvirus such as variola or vaccinia, or molluscum contagiosum), a picornavirus (e.g., rhinovirus or enterovirus), an orthomyxovirus (e.g., influenza virus, avian influenza), a paramyxovirus (e.g., parainfluenza virus, mumps virus, measles virus, and respiratory syncytial virus (RSV), a coronavirus (e.g., SARS), a papovavirus (e.g., papillomaviruses, such as those that cause genital warts, common warts, or plantar warts), hepadnavirus (e.g., hepatitis B virus), a flavivirus (e.g., hepatitis C virus or Dengue virus), or a retrovirus (e.g., a lentivirus such as HIV), ebola virus;

Neoplastic diseases such as bladder cancer, cervical dysplasia, cervical cancer, actinic keratosis, basal cell carcinoma, cutaneous T-cell lymphoma, mycosis fungoides, Sezary Syndrome, HPV associated head and neck cancer (e.g., HPV positive oropharyngeal squamous cell carcinoma), Kaposi's sarcoma, melanoma, squamous cell carcinoma, renal cell carcinoma, acute myeloid leukemia, chronic myeloid leukemia, chronic lymphocytic leukemia, multiple myeloma, Hodgkin's lymphoma, non-Hodgkin's lymphoma, B-cell lymphoma, hairy cell leukemia, esophageal cancer, and other cancers;

T_{H}2-mediated atopic diseases such as atopic dermatitis or eczema, eosinophilia, asthma, allergy, allergic rhinitis, and Omenn's syndrome;

Diseases associated with wound repair, such as, for example, inhibition of keloid formation and other types of scarring (e.g., enhancing wound healing, including chronic wounds); and

Parasitic diseases including but not limited to malaria, leishmaniasis, cryptosporidiosis, toxoplasmosis, and trypanosome infection.

In addition, a compound (particularly one of Formula (II)), salt, or pharmaceutical composition described herein may be used as a vaccine adjuvant for use in conjunction with any material that increases either humoral and/or cell mediated immune responses, such as, for example, tumor antigens (e.g., MAGE-3, NY-ESO-1); live viral, bacterial, or parasitic immunogens; inactivated viral, protozoal, fungal, or bacterial immunogens; toxoids; toxins; polysaccharides; proteins; glycoproteins; peptides; cellular vaccines; DNA vaccines; autologous vaccines; recombinant proteins; and the like.

Examples of vaccines that can benefit from use of a compound (particularly one of Formula (II)), salt, or composition identified herein as a vaccine adjuvant include BCG vaccine, cholera vaccine, plague vaccine, typhoid vaccine, hepatitis A vaccine, hepatitis B vaccine, hepatitis C vaccine, influenza A vaccine, influenza B vaccine, malaria vaccine, parainfluenza vaccine, polio vaccine, rabies vaccine, measles vaccine, mumps vaccine, rubella vaccine, yellow fever vaccine, tetanus vaccine, diphtheria vaccine, hemophilus influenza b vaccine, tuberculosis vaccine, meningococcal and pneumococcal vaccines, adenovirus vaccine, HIV vaccine, chicken pox vaccine, cytomegalovirus vaccine, dengue vaccine, feline leukemia vaccine, fowl plague vaccine, HSV-1 vaccine and HSV-2 vaccine, hog cholera vaccine, Japanese encephalitis vaccine, respiratory syncytial virus vaccine, rotavirus vaccine, papilloma virus vaccine, yellow fever vaccine, ebola virus vaccine.

Compounds (particularly one of Formula (II)), salts, or pharmaceutical compositions identified herein may be particularly useful as vaccine adjuvants when used in conjunction with tumor antigens associated with colorectal cancer, head and neck cancer, breast cancer, lung cancer and melanoma.

Compounds (particularly one of Formula (II)), salts, or pharmaceutical compositions identified herein may be particularly useful in individuals having compromised immune function. For example, compounds, salts, or compositions may be used for treating opportunistic infections and tumors that occur after suppression of cell mediated immunity in, for example, transplant patients, cancer patients, and HIV patients.

One or more of the above diseases or types of diseases, for example, an infectious disease (e.g., a viral, bacterial, fungal, or parasitic infection) or neoplastic disease may be treated in a human or animal in need thereof (having the disease) by administering a therapeutically effective amount of a compound described herein (particularly one of Formula (II)), salt, or composition to the human or animal.

A human or animal may also be vaccinated by administering an effective amount of a compound (particularly one of Formula (II)), salt, or composition described herein as a vaccine adjuvant. In one embodiment, a method of vaccinating a human or animal includes administering an effective amount of a compound described herein (particularly one of Formula (II)), salt, or composition described herein to the human or animal as a vaccine adjuvant. The vaccine adjuvant can be co-administered with the material that increases one or more humoral and cell mediated immune responses by including each in the same composition. Alternatively, the vaccine adjuvant and the material that increases either humoral and/or cell mediated immune responses can be in separate compositions.

Compounds (particularly one of Formula (II)), salts, or compositions identified herein may as prophylactic or therapeutic vaccine adjuvants in veterinary applications. Compounds, salts, or compositions identified herein may be administered to, for example, pigs, horses, cattle, sheep, dogs, cats, poultry (such as chickens or turkeys), etc.

Compounds (particularly one of Formula (II)), salts, or compositions identified herein may be particularly useful when an effective amount is administered to a human or animal to treat bladder cancer, cervical dysplasia, actinic keratosis, basal cell carcinoma, genital warts, herpes virus infection, or cutaneous T-cell lymphoma. For these conditions, administration of the compound, salt, or composition of the disclosure is preferably topical (i.e., applied directly to the surface of a tumor, a lesion, a wart, or an infected tissue, etc.).

In one embodiment, an effective amount of compound, salt, or composition described herein, such as an aqueous composition is administered into the bladder of a human or animal that has at least one tumor of the bladder by intravesical instillation (e.g., administration using a catheter).

An amount of a compound (particularly one of Formula (II)) or salt effective to induce cytokine biosynthesis will typically cause one or more cell types, such as monocytes, macrophages, dendritic cells, and B-cells to produce an amount of one or more cytokines, such as, for example, IFN-alpha, IFN-gamma, TNF-alpha, and IP-10 that is increased (induced) over a background level of such cytokines. The precise dose will vary according to factors known in the art but is typically to be a dose of 100 ng/kg to 50 mg/kg, or 10 μg/kg to 5 mg/kg. In other embodiments, the amount can be, for example, from 0.01 mg/m² to 5.0 mg/m² (computed according to the Dubois method as described above), although in other embodiments the induction of cytokine biosynthesis may be performed by administering a compound or salt in a dose outside this range. In some of these embodiments, the method includes administering sufficient compound or salt or composition to provide a dose from 0.1 mg/m² to 2.0 mg/m² to the subject, for example, a dose of from 0.4 mg/m² to 1.2 mg/m².

A method of treating a viral infection in a human or animal and a method of treating a neoplastic disease in a human or animal can include administering an effective amount of a compound (particularly one of Formula (II)) or salt described herein to the human or animal.

An effective amount to treat or inhibit a viral infection can be an amount that will cause a reduction in one or more of the manifestations of viral infection, such as viral lesions, viral load, rate of virus production, and mortality as compared to untreated humans or animals. The precise amount that is effective for such treatment will vary according to factors known in the art but it is normally a dose of 100 ng/kg to 50 mg/kg, or 10 μg/kg to 5 mg/kg.

An amount of a compound (particularly one of Formula (II)) or salt effective to treat a neoplastic condition can be an amount that causes a reduction in tumor size or in the number of tumor foci. The precise amount will vary according to factors known in the art but is typically 100 ng/kg to 50 mg/kg, or 10 μg/kg to 5 mg/kg. In other embodiments, the amount is typically, for example, from 0.01 mg/m² to 5.0 mg/m² (computed according to the Dubois method as described above), although in some embodiments the induction of cytokine biosynthesis may be performed by administering a compound or salt in a dose outside this range. In some of these embodiments, the method includes administering sufficient compound or salt or composition to provide a dose from 0.1 mg/m² to 2.0 mg/m² to the subject, for example, a dose of from 0.4 mg/m² to 1.2 mg/m².

### EMBODIMENTS

Embodiment 1 is a compound of Formula (I), or salt thereof:

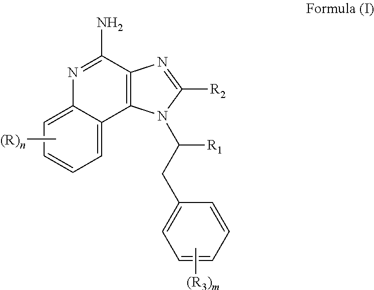

wherein:

m is an integer of 0 or 1;

n is an integer of 0 or 1;

R is selected from the group consisting of halogen, hydroxy, alkyl, alkoxy, and —C(O)—O-alkyl;

R₁ is alkyl or —CH₂—O—C₁₋₄alkyl;

R₂ is selected from the group consisting of hydrogen, methyl, ethyl, n-propyl, n-butyl, —CH₂OCH₃, —CH₂OCH₂CH₃, and —CH₂CH₂OCH₃; and

R₃ is selected from the group consisting of halogen, hydroxy, alkyl, and alkoxy; with the proviso that when R₃ is alkoxy, R₁ is alkyl.

Embodiment 2 is the compound or salt of embodiment 1, which is a compound of Formula (II) or salt thereof:

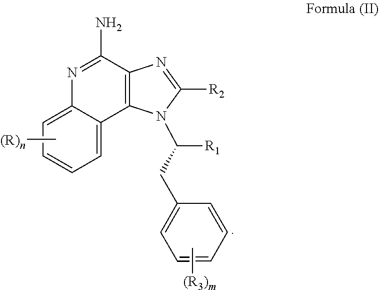

Embodiment 3 is the compound or salt of embodiment 1, which is a compound of Formula (III), or salt thereof:

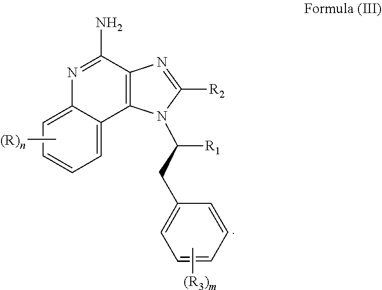

Embodiment 4 is the compound or salt of any of embodiments 1 through 3, wherein m=0.

Embodiment 5 is the compound or salt of any of embodiments 1 through 3, wherein m=1.

Embodiment 6 is the compound or salt of embodiment 5, wherein the —R₃ group is in an ortho, meta, or para position.

Embodiment 7 is the compound or salt of embodiment 6, wherein the —R₃ group is in the para position.

Embodiment 8 is the compound or salt of any of embodiments 5 through 7, wherein R₃ is selected from the group consisting of halogen, hydroxy, —C₁₋₈alkyl, and —C₁₋₈alkoxy.

Embodiment 9 is the compound or salt of embodiment 8, wherein R₃ is —O—C₁₋₈alkyl.

Embodiment 10 is the compound or salt of embodiment 9, wherein R₃ is —O—C₁₋₆alkyl.

Embodiment 11 is the compound or salt of embodiment 10, wherein R₃ is —O—C₁₋₄alkyl.

Embodiment 12 is the compound or salt of embodiment 8, wherein R₃ is selected from the group consisting of halogen, hydroxy, —C₁₋₈alkyl.

Embodiment 13 is the compound or salt of any of embodiments 1 through 12, wherein n is 0.

Embodiment 14 is the compound or salt of any of embodiments 1 through 12, wherein n is 1.

Embodiment 15 is the compound or salt of embodiment 14, wherein R is selected from the group consisting of halogen, hydroxy, —C₁₋₇alkyl, —C₁₋₇alkoxy, and —C(O)—O—C₁₋₅alkyl.

Embodiment 16 is the compound or salt of embodiment 15, wherein R is selected from the group consisting of hydroxy, F, and Cl.

Embodiment 17 is the compound or salt of embodiment 16, wherein R is selected from the group consisting of F and Cl.

Embodiment 18 is the compound or salt of any of embodiments 1 through 17, wherein R₁ is —C₁₋₆alkyl or —CH₂—O—C₁₋₄alkyl; with the proviso that when R₃ is alkoxy, R₁ is —C₁₋₆alkyl.

Embodiment 19 is the compound or salt of embodiment 18, wherein R₁ is —C₁₋₆alkyl.

Embodiment 20 is the compound or salt of embodiment 19, wherein R₁ is —C₁₋₄alkyl.

Embodiment 21 is the compound or salt of embodiment 20, wherein R₁ is —CH₂—O—C₁₋₄alkyl.

Embodiment 22 is the compound or salt of embodiment 21, wherein R₁ is —CH₂—O—CH₃ or —CH₂—O—CH₂CH₃.

Embodiment 23 is the compound or salt of embodiment 22, wherein R₁ is —CH₂—O—CH₃.

Embodiment 24 is the compound or salt of embodiment 22, wherein R₁ is —CH₂—O—CH₂CH₃.

Embodiment 25 is the compound or salt of any of embodiments 1 through 24, wherein R₂ is selected from the group consisting of hydrogen, methyl, and ethyl.

Embodiment 26 is the compound or salt of embodiment 25, wherein R₂ is hydrogen.

Embodiment 27 is the compound or salt of any of embodiments 1 through 3, wherein m is 0; n is 0; R₁ is —C₁₋₆alkyl; and R₂ is selected from the group consisting of hydrogen, methyl, and ethyl.

Embodiment 28 is the compound or salt of embodiment 27, wherein R₁ is —C₁₋₄alkyl; and R₂ is hydrogen.

Embodiment 29 is the compound or salt of embodiment 28, wherein the compound is 1-[(1R)-1-methyl-2-phenyl-ethyl]imidazo[4,5-c]quinolin-4-amine (Example 1).

Embodiment 30 is the compound or salt of embodiment 28, wherein the compound is 1-[(1R)-1-benzylpentyl]imidazo[4,5-c]quinolin-4-amine (Example 2).

Embodiment 31 is the compound or salt of any of embodiments 1 through 3, wherein m is 0; n is 0; R₁ is —CH₂—O—C₁₋₄alkyl; and R₂ is selected from the group consisting of hydrogen, methyl, and ethyl.

Embodiment 32 is the compound or salt of embodiment 31, wherein R₂ is hydrogen.

Embodiment 33 is the compound or salt of embodiment 32, wherein the compound is 1-[(1R)-1-benzyl-2-methoxy-ethyl]imidazo[4,5-c]quinolin-4-amine (Example 3).

Embodiment 34 is the compound or salt of embodiment 32, wherein the compound is 1-[(1S)-1-benzyl-2-methoxy-ethyl]imidazo[4,5-c]quinolin-4-amine (Example 5).

Embodiment 35 is the compound or salt of embodiment 32, wherein the compound is 1-[(1R)-1-benzyl-2-ethoxy-ethyl]imidazo[4,5-c]quinolin-4-amine (Example 4).

Embodiment 36 is the compound or salt of embodiment 32, wherein the compound is 1-[(1S)-1-benzyl-2-ethoxy-ethyl]imidazo [4,5-c]quinolin-4-amine (Example 6).

Embodiment 37 is the compound or salt of any of embodiments 1 through 3, wherein m is 1; n is 0; R₁ is —C₁₋₆alkyl; R₂ is selected from the group consisting of hydrogen, methyl, and ethyl; and R₃ is —O—C₁₋₈alkyl.

Embodiment 38 is the compound or salt of embodiment 37, wherein R₁ is —C₁₋₆alkyl; R₂ is hydrogen; and R₃ is —O—C₁₋₆alkyl.

Embodiment 39 is the compound or salt of embodiment 38, wherein the compound is 1-[(1R)-1-[(4-butoxyphenyl)methyl]pentyl]imidazo [4,5-c]quinolin-4-amine (Example 7).

Embodiment 40 is the compound or salt of any of embodiments 1 through 39, wherein the salt is a pharmaceutically acceptable salt.

Embodiment 41 is the compound or salt of embodiments 40, wherein the pharmaceutically acceptable salt is a hydrochloride salt.

Embodiment 42 is a pharmaceutical composition comprising an effective amount of a compound or salt of any of embodiments 1 through 41 in combination with a pharmaceutically acceptable carrier.

Embodiment 43 is the pharmaceutical composition of embodiment 42, wherein the compound of Formula (II) or salt thereof is present in at least 80% enantiomeric excess.

Embodiment 44 is the pharmaceutical composition of embodiment 43, wherein the compound of Formula (II) or salt thereof is present in at least 90% enantiomeric excess.

Embodiment 45 is the pharmaceutical composition of embodiment 44, wherein the compound of Formula (II) or salt thereof is present in at least 95% enantiomeric excess.

Embodiment 46 is the pharmaceutical composition of embodiment 45, wherein the compound of Formula (II) or salt thereof is present in at least 97% enantiomeric excess. Embodiment 47 is the pharmaceutical composition of embodiment 46, wherein the compound of Formula (II) or salt thereof is present in at least 98% enantiomeric excess.

Embodiment 48 is the pharmaceutical composition of embodiment 47, wherein the compound of Formula (II) or salt thereof is present in at least 99% enantiomeric excess.

Embodiment 49 is the pharmaceutical composition of embodiment 48, wherein the compound of Formula (II) or salt thereof is present in at least 99.5% enantiomeric excess.

Embodiment 50 is the pharmaceutical composition of embodiment 49, wherein the compound of Formula (II) or salt thereof is present in at least 99.8% enantiomeric excess.

Embodiment 51 is the pharmaceutical composition of any of embodiments 42 through 50, further comprising an antigen.

Embodiment 52 is the pharmaceutical composition of any of embodiments 42 through 51 for use in treating an infectious disease in a human or animal.

Embodiment 53 is the pharmaceutical composition of embodiment 52 for use in treating a viral, bacterial, fungal, or parasitic infection in a human or animal. Embodiment 54 is a method of inducing cytokine biosynthesis in a human or animal comprising administering an effective amount of a compound or salt of any of embodiments 1 through 41 to the human or animal.

Embodiment 55 is the method of inducing cytokine biosynthesis of embodiment 54, wherein administering comprises administering an effective amount of a compound or salt of any of embodiments 1, 2, and 4 through 41, as dependent on embodiment 1 or 2, to the human or animal.

Embodiment 56 is the method of inducing cytokine biosynthesis of embodiment 54 or 55, wherein the cytokine is IFN-alpha.

Embodiment 57 is the method of inducing cytokine biosynthesis of embodiment 54 or 55, wherein the cytokine is IFN-gamma.

Embodiment 58 is the method of inducing cytokine biosynthesis of embodiment 54 or 55, wherein the cytokine is TNF-alpha.

Embodiment 59 is the method of inducing cytokine biosynthesis of embodiment 54 or 55, wherein the cytokine is IP-10.

Embodiment 60 is a method of treating a neoplastic disease in a human or animal by administering an effective amount of a compound or salt of any of embodiments 1 through 41.

Embodiment 61 is the method of treating a neoplastic disease of embodiment 60, wherein administering comprises administering an effective amount of a compound or salt of any of embodiments 1, 2, and 4 through 41, as dependent on embodiment 1 or 2, to the human or animal.

Embodiment 62 is the method of embodiment 60 or 61, wherein the neoplastic disease is selected from bladder cancer, cervical dysplasia, cervical cancer, actinic keratosis, basal cell carcinoma, cutaneous T-cell lymphoma, mycosis fungoides, Sezary Syndrome, HPV associated head and neck cancer (e.g., HPV positive oropharyngeal squamous cell carcinoma), Kaposi's sarcoma, melanoma, squamous cell carcinoma, renal cell carcinoma, acute myeloid leukemia, chronic myeloid leukemia, chronic lymphocytic leukemia, multiple myeloma, Hodgkin's lymphoma, non-Hodgkin's lymphoma, B-cell lymphoma, hairy cell leukemia, esophageal cancer, and combinations thereof.

Embodiment 63 is a compound or salt of any of embodiments 1 through 41 for use as a vaccine adjuvant in treating an infectious disease in a human or animal.

Embodiment 64 is a compound or salt of any of embodiments 1, 2, and 4 through 41, as dependent on embodiment 1 or 2, for use as a vaccine adjuvant in treating an infectious disease in a human or animal.

Embodiment 65 is the compound or salt of embodiment 63 or 64, wherein the infectious disease is a viral, bacterial, fungal, or parasitic infection.

Embodiment 66 is the compound or salt of any of embodiments 63 through 65, wherein the treatment is a therapeutic or prophylactic treatment.

### EXAMPLES

Objects and advantages of the disclosure are further illustrated by the examples provided herein. The particular materials and amounts thereof recited in these examples, as well as other conditions and details, are merely illustrative and are not intended to be limiting. The person of ordinary skill in the art, after carefully reviewing the entirety of this disclosure, will be able to use materials and conditions in addition to those specifically described in the examples.

Automated flash chromatography (AFC) was carried out using an ISOLARA HPFC system (an automated high-performance flash purification product available from Biotage Incorporated, Charlottesville, Va.). The eluent used for each purification is given in the example. In some chromatographic separations, the solvent mixture 80/18/2 v/v/v (by volume) chloroform/methanol/concentrated ammonium hydroxide (CMA) was used as the polar component of the eluent. In these separations, CMA was mixed with chloroform in the indicated ratio.

Proton nuclear magnetic resonance (¹H NMR) analysis was conducted using a BRUKER A500 NMR spectrometer (Bruker Corporation, Bilerica, Mass.).

10% Palladium on carbon, 3-chloroperbenzoic acid (57-86%, MCPBA), (2,2,6,6-tetramethylpiperidin-1-yl)oxyl (TEMPO), imidazole, and cesium carbonate were obtained from the Sigma-Aldrich Company, St. Louis, Mo.

Triethyl orthoformate, 3% platinum on carbon, n-propyl acetate, para-toluenesulfonyl chloride, triphenylphosphine, dimethyl sulfate, diethyl sulfate, N-methyl morpholine, 1-bromobutane, pyridine hydrochloride, and tert-butyl N-[(1S)-1-[(4-tert-butoxyphenyl)methyl]-2-hydroxy-ethyl]carbamate (CAS number 66605-57-0) were obtained from the Alfa Aesar Company, Haverhill, Mass..

Isobutyl chloroformate, N-Boc-O-tert-butyl-L-tyrosine (CAS number 47375-34-8), di-tert-butyl dicarbonate, propyltriphenylphosphonium bromide, 11% solution of potassium bis(trimethylsilyl)amide in toluene, and 3-chloroperbenzoic acid (80%, MCPBA) were obtained from Oakwood Products Incorporated, Estill, S.C.

Tert-butyl N-[(1R)-1-[(4-tert-butoxyphenyl)methyl]-2-hydroxy-ethyl]carbamate (CAS number 106454-69-7) was obtained from Combi-Blocks, San Diego Calif.

Iodine was obtained from Mallinckrodt, Inc., St. Louis Mo.

Sodium bromide, potassium iodide, and sodium thiosulfate 0.1 N volumetric solution were obtained from J.T. Baker Chemical Co. Phillipsburg, N.J.

Triethylamine was obtained from EMD Millipore Corporation, Darmstadt Germany.

CLOROX bleach was the source of sodium hypochlorite solution and was obtained from The Clorox Company, Oakland, Calif.. The sodium hypochlorite concentration was determined by titration using iodine and sodium thiosulfate 0.1 N volumetric solution.

### EXAMPLE 1

### 1-[(1R)-1-methyl-2-phenyl-ethyl]imidazo [4,5-c]quinolin-4-amine

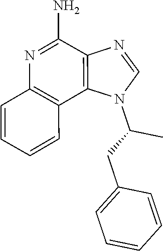 [SMILES: C[C@H](Cc1ccccc1)n1cnc2c(N)nc3ccccc3c21]

### Part A

A 500 mL round bottom flask was charged with triphenylphosphine (5.24 grams (g), 20.0 millimoles (mmol)), imidazole (1.36 g, 20.0 mmol) and 80 milliliters (mL) of methylene chloride. The mixture was stirred until all solids were dissolved and then iodine (5.08 g, 20.0 mmol) was added in small portions and the mixture was stirred for 30 minutes. A solution of tert-butyl N-[(1S)-1-[(4-tert-butoxyphenyl)methyl]-2-hydroxy-ethyl]carbamate (5.02 g, 20.0 mmol) dissolved in 80 mL of methylene chloride was then added dropwise over 30 minutes. After stirring for 3 hours, a 5% solution of aqueous Na₂S₂O₃ (100 mL) was added and the mixture was transferred to a separatory funnel and the layers were separated. The organic portion was sequentially washed with 5% aqueous Na₂S₂O₃, water and brine and the organic portion was dried over Na₂SO₄, filtered and concentrated under reduced pressure to give a pale orange syrup. The syrup was combined with 25% ethyl acetate/hexanes to precipitate triphenylphosphine oxide and the mixture was filtered through a plug of silica gel eluting with 25% ethyl acetate/hexanes. The filtrate was concentrated to give 5.00 g of tert-butyl N-[(1S)-1-benzyl-2-iodo-ethyl]carbamate as a white solid.

### Part B

A solution of tert-butyl N-[(1S)-1-benzyl-2-iodo-ethyl]carbamate (4.32 g, 12.0 mmol) dissolved in 50 mL methanol was placed in a pressure bottle followed by addition of 200 milligrams (mg) of 10% palladium on carbon and 2.5 g of sodium bicarbonate. The bottle was then shaken under an atmosphere of hydrogen (50 pounds per square inch (PSI)) overnight. The reaction mixture was filtered through a pad of CELITE and the filtrate was concentrated under reduced pressure to give an oily syrup. The syrup was dissolved in ethyl acetate and was sequentially washed with 5% aqueous Na₂S₂O₃, water and brine. The organic portion was dried over Na₂SO₄, filtered and concentrated under reduced pressure to give a solid. The solid was concentrated from hexanes to give 2.72 g of tert-butyl N-[(1R)-1-methyl-2-phenyl-ethyl]carbamate as a white powder.

### Part C

A solution tert-butyl N-[(1R)-1-methyl-2-phenyl-ethyl]carbamate (2.72 g, 11.6 mmol) dissolved in 20 mL of ethanol was combined with 2 mL of concentrated hydrochloric acid. The stirred reaction mixture was heated to reflux for 3.5 hours and then concentrated under reduced pressure to give an oil. Crystallization from acetonitrile gave 1.62 of (2R)-1-phenylpropan-2-amine hydrochloride as white crystals.

### Part D

A solution of 4-chloro-3-nitroquinoline (1.75 g, 8.41 mmol) dissolved in 50 mL of methylene chloride was combined with (2R)-1-phenylpropan-2-amine hydrochloride (1.52 g, 8.85 mmol) and triethylamine (3.51 mL, 25.2 mmol) and the reaction mixture was stirred under an atmosphere of nitrogen overnight. The reaction mixture was concentrated to give a yellow solid. The solid was dissolved in 50 mL of ethyl acetate and 50 mL of water and the layers were separated. The organic portion was washed successively with water (2×) and brine. The organic portion was dried over Na₂SO₄, filtered and concentrated to give a yellow syrup. Crystallization from ethyl acetate/hexanes gave 1.90 g of N-[(1R)-1-methyl-2-phenyl-ethyl]-3-nitro-quinolin-4-amine as a yellow syrup.

### Part E

A solution of N-[(1R)-1-methyl-2-phenyl-ethyl]-3-nitro-quinolin-4-amine (1.90 g, 6.18 mmol) dissolved in 50 mL of acetonitrile was placed in a pressure bottle followed by addition of 100 mg of 3% platinum on carbon. The bottle was then shaken under an atmosphere of hydrogen (38 PSI) for 3 hours. The reaction mixture was filtered through a pad of CELITE, rinsing with ethanol, and the filtrate was concentrated under reduced pressure to give 1.70 g of N4-[(1R)-1-methyl-2-phenyl-ethyl]quinoline-3,4-diamine as a white solid.

### Part F

A solution of N4-[(1R)-1-methyl-2-phenyl-ethyl]quinoline-3,4-diamine (1.70 g, 6.13 mmol) dissolved in 50 mL of n-propyl acetate was combined with triethyl orthoformate (3.06 mL, 18.4 mmol) and 100 mg of pyridine hydrochloride and the mixture was heated to 100° C. overnight. The warm reaction mixture was washed successively with saturated NaHCO₃ solution, water and brine. The organic portion was dried over Na₂SO₄, filtered and concentrated to give a yellow syrup. Purification by column chromatography (SiO₂, 1% methanol/chloroform to 5% methanol/chloroform) gave 1.71 g 1-[(1R)-1-methyl-2-phenyl-ethyl]imidazo[4,5-c]quinoline as an amber syrup.

### Part G

A solution of 1-[(1R)-1-methyl-2-phenyl-ethyl]imidazo[4,5-c]quinoline (1.71 g, 5.96 mmol) dissolved in 40 mL of methylene chloride was combined with 1.35 g of MCPBA (80%) and stirred for 60 minutes. The reaction mixture was combined with 10% Na₂CO₃ solution and 10 mL of methylene chloride and the layers were separated. The aqueous portion was further extracted with an additional 10 mL portion of methylene chloride. The combined organic layers were washed with brine and concentrated to give a rust colored foam. A stirred solution of the rust colored foam dissolved in 30 mL of methylene chloride was combined with 8 mL of concentrated NH₄OH solution and p-toluenesulfonyl chloride (1.25 g, 6.56 mmol). After stirring for 55 minutes, the reaction mixture was diluted with water and the layers were separated. The organic portion was washed successively with water (2×) and brine. The organic portion was dried over Na₂SO₄, filtered and concentrated under reduced pressure. Crystallization from acetonitrile gave 1.02 g of 1-[(1R)-1-methyl-2-phenyl-ethyl]imidazo[4,5-c]quinolin-4-amine as copper colored crystals.

¹H NMR (500 MHz, METHANOL-d₄) δ 8.21-8.25 (m, 2H), 7.72 (d, J=8.1 Hz, 1H), 7.52 (t, J=7.6 Hz, 1H), 7.36 (t, J=7.6 Hz, 1H), 7.10-7.21 (m, 3H), 7.03 (m, 2H), 5.57 (m, 1H), 3.35 (m, 2H), 1.75 (d, J=6.6 Hz, 3H).

### EXAMPLE 2

### 1-[(1R)-1-benzylpentyl]imidazo[4,5-c]quinolin-4-amine

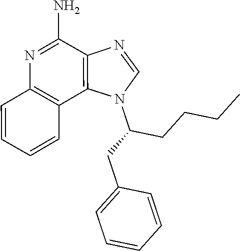 [SMILES: CCCC[C@H](Cc1ccccc1)n1cnc2c(N)nc3ccccc3c21]

### Part A

A solution of tert-butyl N-[(1S)-1-[(4-tert-butoxyphenyl)methyl]-2-hydroxy-ethyl]carbamate (2.51 g, 10.0 mmol) dissolved in 60 mL of a 1:1 mixture of ethyl acetate/toluene was placed in a round bottom flask. A solution of sodium bromide (1.08 g, 10.5 mmol) dissolved in 5 mL of deionized water was then added to the flask and the mixture was stirred in a −2° C. bath. TEMPO (22 mg) was then added to the stirred mixture followed by the dropwise addition of a solution containing aqueous sodium hypochlorite (4.4% by weight, 18.6 g, 11.0 mmol) and NaHCO₃ (2.56 g, 30 mmol) dissolved in 20 mL of deionized water. After addition was complete, the mixture was stirred for an additional 30 minutes. The mixture was then diluted with ethyl acetate (20 mL) and transferred to a separatory funnel and the layers were separated. The aqueous layer was extracted with an additional 20 mL portion of ethyl acetate. The combined organic portions were successively washed with 30 mL of 10% aqueous citric acid containing 360 mg of potassium iodide, 10% aqueous Na₂S₂O₃, water and finally brine. The organic portion was dried over Na₂SO₄, filtered and concentrated to give 2.22 g of tert-butyl N-[(1S)-1-benzyl-2-oxo-ethyl]carbamate as an off-white solid.

### Part B

A dry 250 mL round bottom flask was charged with propyltriphenylphosphonium bromide (3.43 g, 8.92 mmol) and 30 mL of anhydrous toluene. The reaction mixture was cooled in a 0° C. bath and stirred under a nitrogen atmosphere. An 11% solution of potassium bis(trimethylsilyl)amide in toluene (16.1 g, 8.92 mmol) was then added to the flask. After stirring for 15 minutes, the reaction mixture was transferred to a −78° C. bath and a solution of tert-butyl N-[(1S)-1-benzyl-2-oxo-ethyl]carbamate (2.22 g, 8.92 mmol) dissolved in 15 mL of anhydrous toluene was added. The stirred mixture was allowed to warm to ambient temperature overnight. The reaction was quenched by addition of saturated NH₄Cl solution followed by addition of 30 mL of diethyl ether. The layers were separated and the aqueous portion was extracted with an additional 20 mL of diethyl ether. The combined organic layers were washed with brine, dried over MgSO₄, filtered and concentrated under reduced pressure. The resulting material was combined with 25% ethyl acetate/hexanes to precipitate triphenylphosphine oxide which was removed by filtering through a plug of silica gel eluting with 25% ethyl acetate/hexanes. The eluate was concentrated to give a colorless semi-solid. Purification by column chromatography (SiO₂, 10% ethyl acetate/hexanes) gave 1.64 g of tert-butyl N-[(Z,1S)-1-benzylpent-2-enyl]carbamate as a colorless oil which solidified on standing.

### Part C

A solution of tert-butyl N-[(Z,1S)-1-benzylpent-2-enyl]carbamate (1.64 g) dissolved in 25 mL methanol was placed in a pressure bottle followed by addition of 200 mg of 10% palladium on carbon. The bottle was then shaken under an atmosphere of hydrogen (40 PSI) overnight. The reaction mixture was filtered through a pad of CELITE, rinsing with ethanol, and the filtrate was concentrated under reduced pressure to give 1.63 g of tert-butyl N-[(1R)-1-benzylpentyl]carbamate as a colorless solid.

### Part D

A solution of tert-butyl N-[(1R)-1-benzylpentyl]carbamate (1.63 g, 5.87 mmol) dissolved in 20 mL of ethanol was combined with 2 mL of concentrated hydrochloric acid. The stirred reaction mixture was heated to reflux for 2 hours and then concentrated under reduced pressure to give an oil. Crystallization from acetonitrile gave 806 mg of (2R)-1-phenylhexan-2-amine hydrochloride as fluffy white solid.

### Part E

A solution of (2R)-1-phenylhexan-2-amine hydrochloride (806 mg, 3.78 mmol) dissolved in 25 mL of methylene chloride was combined with 4-chloro-3-nitroquinoline (715 mg, 3.44 mmol) and triethylamine (1.44 mL, 10.3 mmol) and the reaction mixture was stirred under an atmosphere of nitrogen overnight. The reaction mixture was concentrated to give a yellow solid. The solid was dissolved in 50 mL of ethyl acetate and washed successively with water (3×) and brine. The organic portion was dried over Na₂SO₄, filtered and concentrated to give a yellow syrup. Purification by column chromatography (SiO₂, 25% ethyl acetate/hexanes) gave 1.18 g of N-[(1R)-1-benzylpentyl]-3-nitro-quinolin-4-amine as a yellow syrup.

### Part F

A solution of N-[(1R)-1-benzylpentyl]-3-nitro-quinolin-4-amine (1.18 g, 3.38 mmol) dissolved in 20 mL of acetonitrile was placed in a pressure bottle followed by addition of 100 mg of 3% platinum on carbon. The bottle was then shaken under an atmosphere of hydrogen (35 PSI) for 4 hours. The reaction mixture was filtered through a pad of CELITE and the filtrate was concentrated under reduced pressure to give 1.07 g of N4-[(1R)-1-benzylpentyl]quinoline-3,4-diamine as a yellow syrup.

### Part G

A solution of N4-[(1R)-1-benzylpentyl]quinoline-3,4-diamine (1.08 g, 3.38 mmol) dissolved in 30 mL of n-propyl acetate was combined with triethyl orthoformate (1.69 mL, 10.2 mmol) and 50 mg of pyridine hydrochloride and the mixture was heated to 100° C. overnight. The warm reaction mixture was washed successively with saturated NaHCO₃ solution, water and brine. The organic portion was dried over Na₂SO₄, filtered and concentrated to give a brown syrup. Purification by column chromatography (SiO₂, 3% methanol/chloroform) gave 1.05 g of 1-[(1R)-1-benzylpentyl]imidazo[4,5-c]quinoline as a yellow syrup.

### Part H

A solution of 1-[(1R)-1-benzylpentyl]imidazo[4,5-c]quinoline (1.05 g, 3.19 mmol) dissolved in 20 mL of methylene chloride was combined with 720 mg of MCPBA (80%) and stirred for 60 minutes. The reaction mixture was combined with 10% Na₂CO₃ solution and 10 mL of methylene chloride and the layers were separated. The aqueous portion was further extracted with two additional 10 mL portions of methylene chloride. The combined organic layers were washed with brine and concentrated to give an amber foam. A stirred solution of the amber foam dissolved in 20 mL of methylene chloride was combined with 5 mL of concentrated NH₄OH solution and p-toluenesulfonyl chloride (670 mg, 3.51 mmol). After stirring for 3 hours, the reaction mixture was diluted with 30 mL of methylene chloride and washed successively with water (3×) and brine. The organic portion was dried over Na₂SO₄, filtered and concentrated under reduced pressure. Purification by column chromatography (SiO₂, 5% methanol/chloroform saturated with NH₄OH) gave a light brown foam. The light brown foam was dissolved in 20 mL of ethanol and 1 mL of concentrated hydrochloric acid. The mixture was evaporated to dryness. Crystallization from acetonitrile gave 353 mg of 1-[(1R)-1-benzylpentyl]imidazo[4,5-c]quinolin-4-amine hydrochloride as light yellow crystals

¹H NMR (500 MHz, METHANOL-d₄) δ 8.61 (s, 1H), 8.33 (d, J=8.2 Hz, 1H), 7.71-7.76 (m, 1H), 7.65-7.71 (m, 1H), 7.54 (dt, J=1.3, 7.7 Hz, 1H), 6.92-7.07 (m, 5H), 5.47-5.59 (m, 1H), 3.48 (dd, J=4.6, 14.1 Hz, 1H), 3.29 (dd, J=9.5, 14.1 Hz, 1H), 2.25 (q, J=7.4 Hz, 2H), 1.33-1.47 (m, 3H), 1.20-1.32 (m, 1H), 0.88 (t, J=7.2 Hz, 3H).

### EXAMPLE 3

### 1-[(1R)-1-benzyl-2-methoxy-ethyl]imidazo[4,5-c]quinolin-4-amine

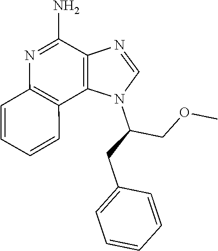 [SMILES: COC[C@@H](Cc1ccccc1)n1cnc2c(N)nc3ccccc3c21]

### Part A

A stirred solution of tert-butyl N-[(1R)-1-[(4-tert-butoxyphenyl)methyl]-2-hydroxy-ethyl]carbamate (2.50 g, 9.95 mmol) and 0.05 g of tetrabutylammonium bromide dissolved in 15 mL of toluene was heated to 60° C. A 50% aqueous sodium hydroxide solution (2.5 g) was then added to the reaction mixture followed by dimethyl sulfate (1.90 g, 15.1 mmol) and stirring was continued overnight. The reaction mixture was quenched with 1 mL of saturated NH₄OH solution. After stirring for 1 hour, water was added to the reaction mixture and the layers were separated. The organic portion was dried over MgSO₄, filtered and concentrated under reduced pressure to give 2.20 g of tert-butyl N-[(1R)-1-benzyl-2-methoxy-ethyl]carbamate as a yellow oil.

### Part B

A mixture of tert-butyl N-[(1R)-1-benzyl-2-methoxy-ethyl]carbamate (2.20 g, 8.30 mmol) in 10 mL of concentrated hydrochloric acid was stirred overnight. The reaction was then combined with 100 mL of methanol and concentrated under reduced pressure to give a white solid. Crystallization from acetonitrile gave 1.53 g of (2R)-1-methoxy-3-phenyl-propan-2-amine hydrochloride as a white crystalline solid.

### Part C

A suspension of (2R)-1-methoxy-3-phenyl-propan-2-amine hydrochloride (1.10 g, 5.47 mmol) and 4-chloro-3-nitroquinoline (1.20 g, 5.77 mmol) in 20 mL of methylene chloride was combined with triethylamine (2.5 mL, 18 mmol) and the reaction mixture was stirred overnight. The reaction mixture was washed with 10% aqueous K₂CO₃ solution and the organic portion was dried over Na₂SO₄, filtered and concentrated under reduced pressure to give a yellow syrup. The yellow syrup was triturated with toluene and filtered to give 2.54 g of material containing N-[(1R)-1-benzyl-2-methoxy-ethyl]-3-nitro-quinolin-4-amine as an orange powder which was used in the next reaction without further purification.

### Part D

A solution of material from the previous reaction (2.54 g) suspended in 100 mL of toluene was placed in a pressure bottle followed by addition of 0.20 g of 5% platinum on carbon. The bottle was then shaken under an atmosphere of hydrogen (50 PSI) overnight. The reaction mixture was filtered through a pad of CELITE, rinsing with methylene chloride, and the filtrate was concentrated under reduced pressure to give 2.27 g of material containing N4-[(1R)-1-benzyl-2-methoxy-ethyl]quinoline-3,4-diamine as an orange oil which was used in the next reaction without further purification.

### Part E

A solution of the material from the previous reaction (2.27 g) dissolved in 100 mL of toluene was combined with triethyl orthoformate (1.65 g, 11.1 mmol) and 100 mg of pyridine hydrochloride and the mixture was heated to 110° C. overnight. The cooled reaction mixture was combined with 10 mL of concentrated NH₄OH solution and the layers were separated. The organic portion was dried over MgSO₄, filtered and concentrated to give 2.40 g of material containing 1-[(1R)-1-benzyl-2-methoxy-ethyl]imidazo[4,5-c]quinoline as a an orange syrup which was used in the next reaction without further purification.

### Part F

A solution of the material from the previous reaction (2.40 g) dissolved in 50 mL of methylene chloride was combined with 2.40 g of MCPBA (77%). After stirring overnight, the reaction mixture was washed with 1% aqueous Na₂CO₃ solution and the organic portion was dried over MgSO₄ and filtered. The resulting solution was transferred to a flask and combined with 15 mL of concentrated NH₄OH solution and benzenesulfonyl chloride (1.35 g, 7.64 mmol) was added to the rapidly stirred reaction mixture. After stirring overnight, the reaction mixture was washed with 5% aqueous K₂CO₃ solution and the organic portion was dried over MgSO₄, filtered and concentrated. Purification by column chromatography (SiO₂, 3% CMA/chloroform-25% CMA/chloroform) gave an amber solid. The solid was heated in refluxing heptane and filtered to remove undissolved solids. The heptane filtrate was allowed to cool and a precipitate formed. The precipitate was isolated by filtration to give 260 mg of an off-white solid. The solid was dissolved in 10 mL of ethanol and 2 mL of concentrated hydrochloric acid solution. After standing for 4 days, crystals formed. The resulting crystals were isolated by filtration, rinsed with cold ethanol and dried with suction to give 60 mg of a fluffy white powder. A portion of the powder was recrystallized from acetonitrile to give of 1-[(1R)-1-benzyl-2-methoxy-ethyl]imidazo[4,5-c]quinolin-4-amine hydrochloride as white needles.

¹H NMR (500 MHz, METHANOL-d₄) δ 8.64 (s, 1H), 8.36 (d, J=8.3 Hz, 1H), 7.67-7.76 (m, 2H), 7.56 (dt, J=1.7, 7.4 Hz, 1H), 7.04-7.18 (m, 5H), 5.73 (m, 1H), 4.01 (d, J=4.6 Hz, 2H), 3.50 (dd, J=5.7, 14.2 Hz, 1H), 3.40 (dd, J=9.3, 14.2 Hz, 1H), 3.39 (s, 1H).

### EXAMPLE 4

### 1-[(1R)-1-benzyl-2-ethoxy-ethyl]imidazo[4,5-c]quinolin-4-amine

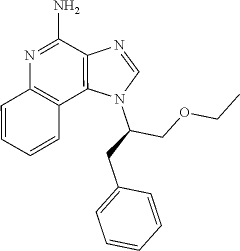 [SMILES: CCOC[C@@H](Cc1ccccc1)n1cnc2c(N)nc3ccccc3c21]

### Part A

A stirred solution of tert-butyl N-[(1R)-1-benzyl-2-hydroxy-ethyl]carbamate (2.50 g, 9.95 mmol) and 0.05 g of tetrabutylammonium bromide dissolved in 15 mL of toluene was combined with a 50% aqueous sodium hydroxide solution (2.5 g) followed by addition of diethyl sulfate (2.30 g, 14.9 mmol). The reaction mixture soon became viscous and an additional 15 mL of toluene was added to maintain stirring. After stirring overnight, an additional portion of diethyl sulfate (2.50 g) was added to the reaction and the mixture was heated to 55° C. for 30 minutes. The reaction mixture was quenched with 3 mL of saturated NH₄OH solution. After stirring for 1 hour, water was added to the reaction mixture and the layers were separated. The organic portion was washed with water and the organic portion was dried over MgSO₄, filtered and concentrated under reduced pressure to give 2.45 g of tert-butyl N-[(1R)-1-benzyl-2-ethoxy-ethyl]carbamate as a yellow oil.

### Part B

A mixture of tert-butyl N-[(1R)-1-benzyl-2-ethoxy-ethyl]carbamate (2.45 g, 8.77 mmol) in 10 mL of concentrated hydrochloric acid was stirred overnight. The reaction was then combined with 100 mL of methanol and concentrated under reduced pressure to give a white solid. Crystallization from ethyl acetate gave 1.67 g of (2R)-1-ethoxy-3-phenyl-propan-2-amine hydrochloride as a white powder.

### Part C

A suspension of (2R)-1-ethoxy-3-phenyl-propan-2-amine hydrochloride (1.41 g, 6.54 mmol) and 4-chloro-3-nitroquinoline (1.45 g, 6.95 mmol) in 20 mL of methylene chloride was combined with triethylamine (3.0 mL, 21.5 mmol) and the reaction mixture was stirred overnight. The reaction mixture was washed with 10% aqueous K2CO3 solution and the organic portion was dried over Na₂SO₄, filtered and concentrated under reduced pressure to give a gummy solid. The gummy solid was triturated with toluene and filtered to give 2.70 g of material containing N-[(1R)-1-benzyl-2-ethoxy-ethyl]-3-nitro-quinolin-4-amine as a yellow powder which was used in the next reaction without further purification.

### Part D

A solution of the material from the previous reaction (2.70 g) suspended in 100 mL of toluene was placed in a pressure bottle followed by addition of 0.2 g of 5% platinum on carbon. The bottle was then shaken under an atmosphere of hydrogen (50 PSI) overnight. The reaction mixture was filtered through a pad of CELITE, rinsing with methylene chloride, and the filtrate was concentrated under reduced pressure to give 2.47 g of material containing N4-[(1R)-1-benzyl-2-ethoxy-ethyl]quinoline-3,4-diamine as an orange syrup which was used in the next reaction without further purification.

### Part E

A solution of the material from the previous reaction (2.47 g) dissolved in 100 mL of toluene was combined with triethyl orthoformate (1.85 g, 12.5 mmol) and 100 mg of pyridine hydrochloride and the mixture was heated to 110° C. overnight. The cooled reaction mixture was combined with 10 mL of concentrated NH₄OH solution and stirred for 1 hour. The mixture was transferred to a separatory funnel and the layers were separated. The organic portion was dried over MgSO₄, filtered and concentrated to give 2.50 g of material containing 1-[(1R)-1-benzyl-2-ethoxy-ethyl]imidazo[4,5-c]quinoline as a an orange syrup which was used in the next reaction without further purification.

### Part F

A solution of the material from the previous reaction (2.50 g) dissolved in 50 mL of methylene chloride was combined with 2.40 g of MCPBA (77%). After stirring overnight, the reaction mixture was washed with 1% aqueous Na₂CO₃ solution and the organic portion was dried over MgSO₄ and filtered. The resulting solution was transferred to a flask and combined with 15 mL of concentrated NH₄OH solution and benzenesulfonyl chloride (1.35 g, 7.64 mmol) was added to the rapidly stirred reaction mixture. After stirring for 4 days, the reaction mixture was washed with 5% aqueous K₂CO₃ solution and the organic portion was dried over MgSO₄, filtered and concentrated. Purification by column chromatography (SiO₂, 10% CMA/chloroform) gave an amber solid. The solid was dissolved in 100 mL of methanol and 5 mL of concentrated hydrochloric acid solution and the mixture was concentrated under reduced pressure. The resulting syrup was concentrated from toluene to give 250 mg of a light-yellow powder. A portion of the resulting material was crystallized from acetonitrile to give 1-[(1R)-1-benzyl-2-ethoxy-ethyl]imidazo[4,5-c]quinolin-4-amine hydrochloride as white needles.

¹H NMR (500 MHz, METHANOL-d₄) δ 8.66 (s, 1H), 8.37 (d, J=8.3 Hz, 1H), 7.67-7.77 (m, 2H), 7.53-7.60 (m, 1H), 7.02-7.19 (m, 5H), 5.73 (m, 1H), 4.05 (d, J=5.1 Hz, 2H), 3.55 (m, 2H), 3.51 (dd, J=5.8, 14.2 Hz, 1H) 3.41 (dd, J=9.2, 14.2 Hz, 1H), 1.13 (t, J=7.0 Hz, 3H).

### EXAMPLE 5

### 1-[(1S)-1-benzyl-2-methoxy-ethyl]imidazo [4,5-c]quinolin-4-amine

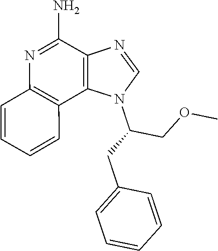 [SMILES: COC[C@H](Cc1ccccc1)n1cnc2c(N)nc3ccccc3c21]

### Part A

A stirred solution of tert-butyl N-[(1S)-1-benzyl-2-hydroxy-ethyl]carbamate (2.50 g, 9.95 mmol) and 0.05 g of tetrabutylammonium bromide dissolved in 75 mL of heptane was heated to 60° C. A 50% aqueous sodium hydroxide solution (3.0 g) was then added to the reaction mixture followed by dimethyl sulfate (1.90 g, 15.1 mmol). After stirring for 60 minutes, an additional portion of dimethyl sulfate (0.45 g) was added to the reaction and stirring was continued at 60° C. overnight. The reaction mixture was quenched with 15 mL of saturated NH₄OH solution. After stirring for 1 hour, water was added to the reaction mixture and the layers were separated. The organic portion was dried over Na₂SO₄, filtered and concentrated under reduced pressure to give 2.52 g of tert-butyl N-[(1S)-1-benzyl-2-methoxy-ethyl]carbamate as a colorless oil.

### Part B

A mixture of tert-butyl N-[(1S)-1-benzyl-2-methoxy-ethyl]carbamate (2.52 g) in 10 mL of concentrated hydrochloric acid was stirred 3 days. The reaction was then combined with 50 mL of methanol and concentrated under reduced pressure to give a white solid. Crystallization from acetonitrile gave 1.57 g of (2S)-1-methoxy-3-phenyl-propan-2-amine hydrochloride as a white crystalline solid.

### Part C

A suspension of (2S)-1-methoxy-3-phenyl-propan-2-amine hydrochloride (1.57 g, 7.78 mmol) and 4-chloro-3-nitroquinoline (1.70 g, 8.15 mmol) in 50 mL of methylene chloride was combined with triethylamine (5.0 mL, 36 mmol) and the reaction mixture was stirred overnight. The reaction mixture was washed with 10% aqueous K₂CO₃ solution and the organic portion was dried over MgSO₄, filtered and concentrated under reduced pressure to give a yellow oil. The yellow oil was triturated with toluene and filtered to give 2.77 g of material containing N-[(1S)-1-benzyl-2-methoxy-ethyl]-3-nitro-quinolin-4-amine as an orange powder which was used in the next reaction without further purification.

### Part D

A solution of the material from the previous reaction (2.62 g) suspended in 100 mL of toluene was placed in a pressure bottle followed by addition of 0.25 g of 5% platinum on carbon. The bottle was then shaken under an atmosphere of hydrogen (50 PSI) overnight. The reaction mixture was filtered through a pad of CELITE, rinsing with methylene chloride, and the filtrate was concentrated under reduced pressure to give 2.60 g of material containing N4-[(1S)-1-benzyl-2-methoxy-ethyl]quinoline-3,4-diamine as an orange syrup which was used in the next reaction without further purification.

### Part E

A solution of the material from the previous reaction (2.38 g) dissolved in 100 mL of toluene was combined with triethyl orthoformate (1.70 g, 11.5 mmol) and 100 mg of pyridine hydrochloride and the mixture was heated to 110° C. overnight. The cooled reaction mixture was washed with 5% aqueous K₂CO₃ solution and the organic portion was dried over MgSO₄, filtered and concentrated to give 2.35 g of material containing 1-[(1S)-1-benzyl-2-methoxy-ethyl]imidazo[4,5-c]quinoline as a an orange syrup which was used in the next reaction without further purification.

### Part F

A solution of the material from the previous reaction (2.35 g) dissolved in 50 mL of methylene chloride was combined with 2.35 g of MCPBA (77%). After stirring overnight, 15 mL of concentrated NH₄OH solution and benzenesulfonyl chloride (1.35 g, 7.64 mmol) were added to the rapidly stirred reaction mixture. After stirring overnight, the reaction mixture was washed with 5% aqueous K₂CO₃ solution and the organic portion was dried over MgSO₄, filtered and concentrated. Purification by column chromatography (SiO₂, 6% CMA/chloroform-16% CMA/chloroform) gave an amber solid. The solid was dissolved in 10 mL of 1.25 N methanolic hydrochloric acid solution and concentrated under reduced pressure. The resulting solid was transferred to a Soxhlet thimble and extracted with refluxing toluene to remove colored impurities yielding 1.00 g of a white solid. A portion of the white solid was crystallized from acetonitrile to give 1-[(1S)-1-benzyl-2-methoxy-ethyl]imidazo[4,5-c]quinolin-4-amine hydrochloride as white needles.

¹H NMR (500 MHz, METHANOL-d₄) δ 8.65 (s, 1H), 8.35 (d, J=8.31 Hz, 1H), 7.66-7.77 (m, 2H), 7.52-7.60 (m, 1H), 7.00-7.19 (m, 5H), 5.73 (m, 1H), 4.01 (d, J=4.5 Hz, 2H), 3.50 (dd, J=5.7, 14.2 Hz, 1H), 3.40 (dd, J=9.3, 14.2 Hz, 1H), 3.39 (s, 3H).

### EXAMPLE 6

### 1-[(1S)-1-benzyl-2-ethoxy-ethyl]imidazo[4,5-c]quinolin-4-amine

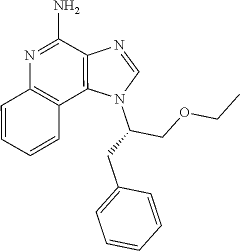 [SMILES: CCOC[C@H](Cc1ccccc1)n1cnc2c(N)nc3ccccc3c21]

### Part A

A stirred solution of tert-butyl N-[(1S)-1-benzyl-2-hydroxy-ethyl]carbamate (2.50 g, 9.95 mmol) and 0.05 g of tetrabutylammonium bromide dissolved in 15 mL of toluene was combined with a 50% aqueous sodium hydroxide solution (2.5 g) followed by addition of diethyl sulfate (2.30 g, 14.9 mmol) and the mixture was heated to 60° C. After stirring for 8 hours, the reaction mixture was cooled. The reaction mixture was transferred to a separatory funnel and washed with water. The organic portion was dried over MgSO₄, filtered and concentrated under reduced pressure to give 2.44 g of tert-butyl N-[(1S)-1-benzyl-2-ethoxy-ethyl]carbamate as a colorless oil.

### Part B

A mixture of tert-butyl N-[(1S)-1-benzyl-2-ethoxy-ethyl]carbamate (2.44 g) in 10 mL of concentrated hydrochloric acid was stirred overnight. The reaction was then combined with 50 mL of ethanol and concentrated under reduced pressure to give a white solid. Crystallization from acetonitrile gave 1.66 g of (2S)-1-ethoxy-3-phenyl-propan-2-amine hydrochloride as white crystals.

### Part C

A suspension of (2S)-1-ethoxy-3-phenyl-propan-2-amine hydrochloride (1.66 g, 7.70 mmol) and 4-chloro-3-nitroquinoline (1.70 g, 8.15 mmol) in 50 mL of methylene chloride was combined with triethylamine (5.0 mL, 36 mmol) and the reaction mixture was stirred overnight. The reaction mixture was washed with 10% aqueous K₂CO₃ solution and the organic portion was dried over MgSO₄, filtered and concentrated under reduced pressure to give a yellow oil. The yellow oil was triturated with toluene and filtered to give 3.06 g of material containing N-[(1S)-1-benzyl-2-ethoxy-ethyl]-3-nitro-quinolin-4-amine as a yellow powder which was used in the next reaction without further purification.

### Part D

A solution of the material from the previous reaction (3.06 g) suspended in 100 mL of toluene was placed in a pressure bottle followed by addition of 0.25 g of 5% platinum on carbon. The bottle was then shaken under an atmosphere of hydrogen (50 PSI) overnight. The reaction mixture was filtered through a pad of CELITE, rinsing with methanol, and the filtrate was concentrated under reduced pressure to give 2.83 g of material containing N4-[(1S)-1-benzyl-2-ethoxy-ethyl]quinoline-3,4-diamine as an orange syrup which was used in the next reaction without further purification.

### Part E

A solution of the material from the previous reaction (2.83 g) dissolved in 150 mL of toluene was combined with triethyl orthoformate (1.80 g, 12.1 mmol) and 100 mg of pyridine hydrochloride and the mixture was heated to 110° C. overnight. The cooled reaction mixture was combined with 5% aqueous K₂CO₃ solution the mixture was transferred to a separatory funnel and the layers were separated. The organic portion was dried over MgSO₄, filtered and concentrated to give 2.58 g of material containing 1-[(1S)-1-benzyl-2-ethoxy-ethyl]imidazo[4,5-c]quinoline as an orange syrup which was used in the next reaction without further purification.

### Part F

A solution of the material from the previous reaction (2.58 g) dissolved in 50 mL of methylene chloride was combined with 2.45 g of MCPBA (77%). After stirring overnight, the reaction mixture was combined with 15 mL of concentrated NH₄OH solution and benzenesulfonyl chloride (1.40 g, 7.93 mmol) was added to the rapidly stirred reaction mixture. After stirring overnight, the reaction mixture was washed with 10% aqueous K₂CO₃ solution and the organic portion was dried over MgSO₄, filtered and concentrated. Purification by column chromatography (SiO₂, 6% CMA/chloroform-16% CMA/chloroform) gave an amber solid. The solid was dissolved in 10 mL of 1.25 N methanolic hydrochloric acid solution and concentrated under reduced pressure. The resulting solid was transferred to a Soxhlet thimble and extracted with refluxing toluene to remove colored impurities yielding 0.74 g of an off-white solid. A portion of the solid was crystallized from acetonitrile to give 1-[(1S)-1-benzyl-2-ethoxy-ethyl]imidazo[4,5-c]quinolin-4-amine hydrochloride as white needles.

¹H NMR (500 MHz, METHANOL-d₄) δ 8.66 (s, 1H), 8.37 (d, J=8.1 Hz, 1H), 7.67-7.77 (m, 2H), 7.55 (m, 1H), 7.01-7.22 (m, 5H), 5.65-5.79 (m, 1H), 4.05 (d, J=5.1 Hz, 2H), 3.55 (m, 2H), 3.51 (dd, J=5.8, 14.2 Hz, 1H) 3.42 (dd, J=9.2, 14.2 Hz, 1H), 1.13 (t, J=6.97 Hz, 3H).

### EXAMPLE 7

### 1-[(1R)-1-[(4-butoxyphenyl)methyl]pentyl]imidazo [4,5-c]quinolin-4-amine

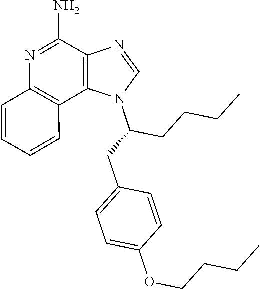 [SMILES: CCCCOc1ccc(C[C@@H](CCCC)n2cnc3c(N)nc4ccccc4c32)cc1]

### Part A

A stirred solution of N-Boc-O-tert-butyl-L-tyrosine (5.00 g, 14.8 mmol) dissolved in 15 mL of anhydrous tetrahydrofuran was cooled to −15° C. in an ice/methanol bath. The solution was combined with N-methyl morpholine (1.63 mL, 14.8 mmol) followed by addition of isobutyl chloroformate (1.92 mL, 14.8 mmol). After stirring for 5 min, the reaction mixture was filtered, rinsing with small portions tetrahydrofuran, to remove N-methyl morpholine hydrochloride. The resulting filtrate was returned to the cold bath and a solution of 1.12 g of NaBH₄ dissolved in 7 mL of H₂O was added over several minutes. After stirring for 90 minutes, the reaction mixture was combined with 75 mL of H₂O followed by addition of 100 mL of ethyl acetate. The layers were separated and the aqueous layer was extracted with an additional 25 mL of ethyl acetate. The combined organic portions were washed with water and brine, dried over Na₂SO₄, filtered and concentrated to give a colorless syrup. The syrup was concentrated from heptanes to give 4.72 g of tert-butyl N-[(1S)-1-[(4-tert-butoxyphenyl)methyl]-2-hydroxy-ethyl]carbamate as a white solid.

### Part B

A solution of tert-butyl tert-butyl N-[(1S)-1-[(4-tert-butoxyphenyl)methyl]-2-hydroxy-ethyl]carbamate (4.72 g, 14.6 mmol) dissolved in 90 mL of a 1:1 mixture of ethyl acetate/toluene was placed in a round bottom flask. A solution of sodium bromide (1.58 g, 15.3 mmol) dissolved in 7.5 mL of deionized water was then added to the flask and the mixture was stirred in a −10° C. bath. TEMPO (33 mg) was then added to the stirred mixture followed by the dropwise addition of a solution containing aqueous sodium hypochlorite (4.4% by weight, 27.2 g, 16.1 mmol) and NaHCO₃ (3.70 g, 43.8 mmol) dissolved in 20 mL of deionized water. After addition was complete, the mixture was stirred for an additional 30 minutes. The mixture was then diluted with ethyl acetate (20 mL) and transferred to a separatory funnel and the layers were separated. The aqueous layer was extracted with an additional 20 mL portion of ethyl acetate. The combined organic portions were successively washed with 30 mL of 10% aqueous citric acid containing 540 mg of potassium iodide, 10% aqueous Na₂S₂O₃, water and finally brine. The organic portion was dried over Na₂SO₄, filtered and concentrated to give 4.69 g of tert-butyl N-[(1S)-1-[(4-tert-butoxyphenyl)methyl]-2-oxo-ethyl]carbamate as an off-white solid.

### Part C

A dry 500 mL round bottom flask was charged with propyltriphenylphosphonium bromide (5.62 g, 14.6 mmol) and 60 mL of anhydrous toluene. The reaction mixture was cooled in a 0° C. bath and stirred under a nitrogen atmosphere. An 11% solution of potassium bis(trimethylsilyl)amide in toluene (26.4 g, 14.6 mmol) was then added to the flask. After stirring for 15 minutes the reaction mixture was transferred to a −78° C. bath and a solution of tert-butyl N-[(1S)-1-[(4-tert-butoxyphenyl)methyl]-2-oxo-ethyl]carbamate (4.69 g, 14.6 mmol) dissolved in 30 mL of anhydrous toluene was added. The stirred mixture was allowed to warm to ambient temperature overnight. The reaction was quenched by addition of saturated NH₄Cl solution followed by addition of 50 mL of diethyl ether. The layers were separated and the aqueous portion was extracted with an additional 20 mL of diethyl ether. The combined organic layers were washed with brine, dried over MgSO₄, filtered and concentrated under reduced pressure. The resulting material was combined with 25% ethyl acetate/hexanes to precipitate triphenylphosphine oxide which was removed by filtering through a plug of silica gel eluting with 25% ethyl acetate/hexanes. The eluate was concentrated to give an amber syrup. Purification by column chromatography (SiO₂, 3% ethyl acetate/hexanes to 20% ethyl acetate/hexanes) gave 3.52 g of tert-butyl N-[(Z,1S)-1-[(4-tert-butoxyphenyl)methyl]pent-2-enyl]carbamate as a light amber syrup.

### Part D

A solution of tert-butyl tert-butyl N-[(Z,1S)-1-[(4-tert-butoxyphenyl)methyl]pent-2-enyl]carbamate (3.52 g) dissolved in 40 mL methanol was placed in a pressure bottle followed by addition of 200 mg of 10% palladium on carbon. The bottle was then shaken under an atmosphere of hydrogen (60 PSI) overnight. The reaction mixture was filtered through a pad of CELITE, rinsing with methanol, and the filtrate was concentrated under reduced pressure to give 3.30 g of tert-butyl N-[(1R)-1-[(4-tert-butoxyphenyl)methyl]pentyl]carbamate as a colorless solid.

### Part E

A solution of tert-butyl N-[(1R)-1-[(4-tert-butoxyphenyl)methyl]pentyl]carbamate (3.30 g, 9.46 mmol) in 30 mL of ethanol was combined with 4 mL of concentrated hydrochloric acid and the mixture was heated to reflux for 2.5 hours. The reaction was then concentrated under reduced pressure to give a colorless syrup. The syrup was again concentrated from ethanol and then from acetonitrile to give 2.17 g of 4-[(2R)-2-aminohexyl]phenol hydrochloride as a white foam.

### Part F

A solution of 4-[(2R)-2-aminohexyl]phenol hydrochloride (2.17 g, 9.49 mmol) in 50 mL of methylene chloride was combined with triethylamine (3.76 mL, 27.0 mmol) followed by the addition of 4-chloro-3-nitroquinoline (1.87 g, 9.00 mmol) and the reaction mixture was stirred under an atmosphere of nitrogen overnight. The reaction mixture was concentrated to give a yellow solid. The solid was triturated with hot water and filtered to give a yellow solid. The yellow solid was dissolved in 10% methanol/chloroform and washed with brine. The organic portion was dried over Na₂SO₄, filtered and concentrated to give 3.09 g of 4-[(2R)-2-[(3-nitro-4-quinolyl)amino]hexyl]phenol as a yellow solid.

### Part G

A suspension of 4-[(2R)-2-[(3-nitro-4-quinolyl)amino]hexyl]phenol (3.09 g, 8.46 mmol) dissolved in 120 mL of a 1:1 mixture of acetonitrile and toluene was placed in a pressure bottle followed by addition of 300 mg of 3% platinum on carbon. The bottle was then shaken under an atmosphere of hydrogen (50 PSI) overnight. The reaction mixture was filtered through a pad of CELITE and the filtrate was concentrated under reduced pressure to give 2.84 g of 4-[(2R)-2-[(3-amino-4-quinolyl)amino]hexyl]phenol as an orange foam.

### Part H

A solution of 4-[(2R)-2-[(3-amino-4-quinolyl)amino]hexyl]phenol (2.84 g, 8.48 mmol) dissolved in 50 mL of n-propyl acetate was combined with triethyl orthoformate (4.22 mL, 25.4 mmol) and 100 mg of pyridine hydrochloride and the mixture was heated to 100° C. overnight. The warm reaction mixture was washed successively with saturated NaHCO₃ solution, water (3×) and brine. The organic portion was dried over Na₂SO₄, filtered and concentrated to give a brown syrup. Purification by column chromatography (SiO2, 1% methanol/chloroform to 10% methanol/chloroform) gave 2.48 g of 4-[(2R)-2-imidazo[4,5-c]quinolin-1-ylhexyl]phenol as an amber foam.

### Part I

To a stirred solution of 4-[(2R)-2-imidazo[4,5-c]quinolin-1-ylhexyl]phenol (1.24 g, 3.59 mmol) dissolved in 10 mL of anhydrous DMF were added Cs₂CO₃ (1.75 g, 5.39 mmol) followed by 1-bromobutane (426 microliters, 3.95 mmol). The reaction mixture was heated to 65° C. under an atmosphere of nitrogen. After 5 hours, the reaction mixture was concentrated under reduced pressure and the resulting syrup was dissolved in 50 mL of ethyl acetate and washed successively with water (4×) and brine. The organic portion was dried over Na₂SO₄, filtered and concentrated. Purification by column chromatography (SiO₂, 1% methanol/chloroform −5% methanol/chloroform) gave 1.24 g of 1-[(1R)-1-[(4-butoxyphenyl)methyl]pentyl]imidazo [4,5-c]quinoline as an amber syrup.

### Part J

A solution of 1-[(1R)-1[(4-butoxyphenyl)methyl]pentyl]imidazo[4,5-c]quinoline (1.24 g, 3.09 mmol) dissolved in 20 mL of methylene chloride was combined with 698 mg of MCPBA (80%) and stirred for 60 minutes. A 10% solution of Na₂CO₃ (10 mL) was then added and the layers were separated. The aqueous portion was extracted with an additional 10 mL portion of methylene chloride. The combined organic portions were washed with brine and concentrated under reduced pressure. The resulting material was dissolved in 30 mL of methylene chloride and combined with 6 mL of concentrated ammonium hydroxide solution and para-toluenesulfonyl chloride (648 mg, 3.40 mmol). After stirring rapidly for 55 min, the reaction mixture was diluted with 10 mL of methylene chloride and washed successively with water (3×) and brine. The organic portion was dried over Na₂SO₄, filtered and concentrated under reduced pressure. Purification by column chromatography (SiO₂, 1% methanol/chloroform −7.5% methanol/chloroform) gave a brown syrup. The brown syrup was dissolved in 15 mL of ethanol and 0.5 mL of concentrated hydrochloric acid and the mixture was concentrated under reduced pressure. Crystallization from acetonitrile gave a solid which was isolated by filtration, rinsed with cold acetonitrile and dried under reduced pressure to give 279 mg of 1-[(1R)-1-[(4-butoxyphenyl)methyl]pentyl]imidazo[4,5-c]quinolin-4-amine hydrochloride as a tan powder.

¹H NMR (500 MHz, METHANOL-d₄) δ 8.56 (s, 1H), 8.32 (d, J=8.2 Hz, 1H), 7.72-7.76 (m, 1H), 7.66-7.71 (m, 1H), 7.54 (t, J=7.5 Hz, 1H), 6.87 (d, J=8.4 Hz, 2H), 6.56 (d, J=8.6 Hz, 2H), 5.40-5.52 (m, 1H), 3.74 (t, J=6.4 Hz, 2H), 3.39 (dd, J=4.2, 14.1 Hz, 1H), 3.21 (dd, J=9.3, 14.2 Hz, 1H), 2.17-2.27 (m, 2H), 1.55-1.66 (m, 2H), 1.33-1.46 (m, 5H), 1.23-1.32 (m, 1H), 0.93 (t, J=7.4 Hz, 3H), 0.88 (t, J=7.1 Hz, 3H)

### Comparative Example 1

### 1-phenethyl-1H-imidazo[4,5-c]quinolin-4-amine

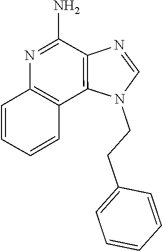 [SMILES: Nc1nc2ccccc2c2c1ncn2CCc1ccccc1]

Comparative Example 1 (CAS Number 99011-69-5) was prepared as described in U.S. Pat. No. 4,689,338 (Gerster and Weeks) and in Gerster et al. J. Med. Chem. 2005, 48 (10), 3481-3491.

### Cytokine Induction in Human Cells

Whole blood was obtained from healthy human donors and collected by venipuncture into vacutainer tubes or syringes containing ethylene diamine tetraacetic acid (EDTA). Human peripheral blood mononuclear cells (PBMC) were purified from the whole blood by density gradient centrifugation. Histopaque 1077 (15 mL, Sigma, St. Louis, Mo.) was transferred to 6×50 mL sterile polypropylene conical tubes. The Histopaque was overlayed with 15-25 mL of blood diluted 1:2 in Hank's Balanced Salts Solution (HBSS) (Gibco, Life Technologies, Grand Island, N.Y.). The tubes were then centrifuged at 1370 revolutions per minute (rpm) for 30 minutes at 20° C., with no brake (400×g, GH 3.8A Rotor).

The interface (buffy coat) containing the PBMC was collected and placed in a new sterile 50 mL conical polypropylene centrifuge tube. The PBMC were mixed with an equal volume of HBSS (about 20 mL from the interface and about 20 mL of HBSS), and then centrifuged at 1090 rpm, 10 minutes, 20° C., with brake (270×g, GH 3.8A Rotor). After completing centrifugation, the cells were resuspended in 2-3mL ACK Red blood cell lysis buffer (ammonium chloride potassium solution, Gibco, Life Technologies) and incubated for 2-5 minutes at 20° C. Next, HBSS (40 mL) was added to the cells, and the sample was centrifuged at 270×g for 10 minutes at 20° C. The supernatant was decanted, and the cell pellet was resuspended in 5 mL AIM V Medium (Gibco, Life Technologies). Cell aggregates and debris were removed by filtering the cell solution through a BD Falcon 70 micron nylon cell strainer (BD Biosciences, San Jose, Calif.).

The number of viable cells was determined by counting with a Miltenyi FACS instrument (Miltenyi Biotec Inc., San Diego, Calif.) or by using a hemacytometer. For determining cell viability with a hemacytometer, the cells were diluted 1/10 in 0.4% trypan blue and HBSS (specifically, 50 microliter of trypan blue +40 microliter of HBSS +10 microliter of cell solution were added to a microfuge tube and mixed). Ten microliters of the diluted cells were then applied to the hemacytometer, and the number of viable PBMC were determined by microscopy.

The PBMC sample was then resuspended in 96-well plates at a concentration of 8×10⁵ cells/well in 0.1 mL of AIM-V medium. Each compound was solubilized in dimethyl sulfoxide (DMSO) to create a 3 mM stock solution. The stock solution was then further diluted with AIM-V medium to prepare the serial dilutions. The diluted compound (100 microliters) was then transferred to the PBMCs to produce testing sets with final compound concentrations of 30, 10, 3.3, 1.1, 0.37, 0.12, 0.04, 0.01 micromolar. The plates also had both positive and negative controls. The negative control wells contained only AIM-V medium with no example compound. The positive control wells contained a control set of imiquimod serially diluted to concentrations of 30, 10, 3.3, 1.1, 0.37, 0.12, 0.04, 0.01 micromolar. The concentrations used in the control set were selected to match the concentrations used in the testing set. The plates were then cultured at 37° C./5% CO₂ for 21-24 hours. Cell-free supernatants were harvested by centrifuging the 96-well plates at 2100 rpm, 23° C. for 10 minutes. Approximately 160 microliters of the supernatant was then stored in a NUNC 96-well plate, covered with the compression cap and stored at −80° C. until the cytokine analysis was done.

IFN-alpha cytokine levels (picograms/mL) were measured by ELISA (human IFN-alpha, pan specific, Mabtech, Cincinnati, Ohio). IFN-gamma and TNF-alpha levels (picograms/mL) were measured by multiplex bead assay (magnetic beads, R & D Systems, Minneapolis, Minn.) according to the manufacturer's instructions.

The data was analyzed to determine the minimum effective concentration (MEC) for each compound at which induction of a particular cytokine was observed in the assay. Specifically, the minimum effective concentration of each compound (micromolar) was determined as the lowest concentration of the compound that induced a measured cytokine response at a level (pictograms/mL) that was at least 2× greater than that observed with the negative control wells. The results are presented in Table 11. The designation “≤0.01” indicate that cytokine induction was observed at the lowest concentration of compound evaluated in the assay.

TABLE 11
Cytokine Induction

| | MEC to Induce Cytokine (micromolar) | | |
|---|---|---|---|
| Compound | IFN-alpha | IFN-gamma | TNF-alpha |
| Comparative Example 1 | 1.1 | >30 | 1.1 |
| Example 1 | 0.37 | 1.1 | 0.12 |
| Example 2 | ≤0.01 | ≤0.01 | ≤0.01 |
| Example 3 | 1.1 | >30 | 1.1 |
| Example 4 | 0.37 | >30 | 0.37 |
| Example 5 | ≤0.01 | ≤0.01 | ≤0.01 |
| Example 6 | ≤0.01 | ≤0.01 | ≤0.01 |
| Example 7 | ≤0.01 | ≤0.01 | ≤0.01 |

### TLR Activation and Specificity

HEK-BLUE-hTLR7 or hTLR8 reporter cells were obtained from InvivoGen, San Diego, Calif. According to the manufacturer's description, these reporter cells were prepared by co-transfection of HEK293 cells with an inducible secreted embryonic alkaline phosphatase (SEAP) reporter gene and either the human TLR7 or TLR8 gene. The SEAP reporter gene was placed under the control of an IFN-β minimal promoter fused to five NF-κB and AP-1-binding sites. In the presence of a TLR ligand, activation of NF-κB and AP-1 occurs, resulting in a corresponding increase in SEAP levels.

Parental HEK293 cells (null), which expressed the inducible SEAP reporter, but did not express TLR7 or TLR8, were obtained from InvivoGen and served as the negative control in the assay.

In the assay, the HEK cells were grown and maintained using standard cell culture techniques in a growth medium that contained Dulbecco's Modified Eagle Medium (ThermoFisher Scientific Incorporated, Waltham, Mass.) supplemented with 1% penicillin/streptomycin and 10% heat-inactivated Gibco fetal bovine serum (ThermoFisher Scientific). Each compound was solubilized in DMSO to create a 3 millimole (mM) stock solution. The stock solution was then further diluted with the growth medium to prepare serial dilutions. Each test compound was tested at a concentration of 30, 10, 3.3, 1.1, 0.37, 0.12, 0.04, and 0.01 micromolar using a 96-well format with 5×10⁴ cells and 200 microliters of growth medium per well.

For each compound, hTLR7, hTLR8, and their respective null control HEK cells were screened. DMSO serially diluted into the growth medium served as the vehicle control. Cell culture supernatants containing the SEAP reporter were collected after an incubation period of 16-20 hours in a cell culture incubator (37° C. and 5% CO₂), and either analyzed immediately or stored at −80° C. SEAP levels were measured using the colorimetric enzyme assay (QUANTI-BLUE (InvivoGen) according to manufacturer's instructions.

The data was analyzed to determine the minimum effective concentration (MEC) for each compound at which activation was observed in the assay. Specifically, the minimum effective concentration of each compound (micromolar) was determined as the lowest concentration of the compound that produced a SEAP expression response at least 2× greater than that observed with the vehicle control wells. The results are presented in Table 12. The designation “≤0.01” indicates that TLR activation was observed at the lowest concentration of compound evaluated in the assay.

TABLE 12
TLR Activation

| | MEC to Produce TLR | |
|---|---|---|
| | Activation (micromolar) | |
| Compound | TLR 7 | TLR 8 |
| Comparative Example 1 | 1.1 | >30 |
| Example 1 | 0.37 | >30 |
| Example 2 | ≤0.01 | 10 |
| Example 3 | 1.1 | >30 |
| Example 4 | 1.1 | >30 |
| Example 5 | ≤0.01 | 10 |
| Example 6 | ≤0.01 | 3.3 |
| Example 7 | ≤0.01 | 10 |

The complete disclosures of the patents, patent documents, and publications cited herein are incorporated by reference in their entirety as if each were individually incorporated. Various modifications and alterations to this invention will become apparent to those of ordinary skill in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. A compound of Formula (I), or salt thereof: 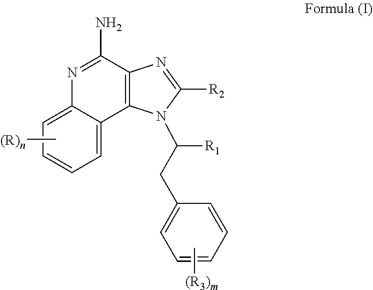
wherein:
m is an integer of 0 or 1;
n is an integer of 0 or 1;
R is selected from the group consisting of halogen, hydroxy, alkyl, alkoxy, and —C(O)—O-alkyl;
R₁ is alkyl or —CH₂—O—C₁₋₄alkyl;
R₂ is selected from the group consisting of hydrogen, methyl, ethyl, n-propyl, n-butyl, —CH₂OCH₃, —CH₂OCH₂CH₃, and —CH₂CH₂OCH₃; and
R₃ is selected from the group consisting of halogen, hydroxy, alkyl, and alkoxy; with the proviso that when R₃ is alkoxy, R₁ is alkyl.

2. The compound or salt of claim 1, which is a compound of Formula (II), or salt
thereof:

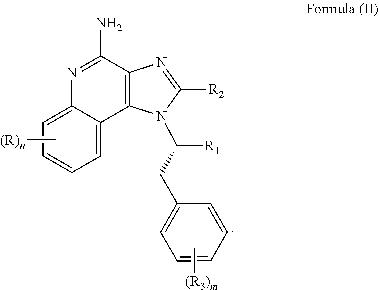

3. The compound or salt of claim 1, which is a compound of Formula (III), or salt
thereof:

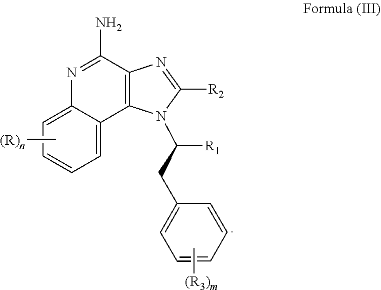

4. The compound or salt of claim 1, wherein
m=0.

5. The compound or salt of claim 1, wherein
m=1.

6. The compound or salt of claim 5, wherein the —R₃ group is in the para position.

7. The compound or salt of claim 5, wherein R₃ is selected from the group consisting of halogen, hydroxy, —C₁₋₈alkyl, and —C₁₋₈alkoxy.

8. The compound or salt of claim 7, wherein R₃ is —O—C₁₋₈alkyl.

9. The compound or salt of claim 1, wherein
n is 0.

10. The compound or salt of claim 1, wherein R₁ is —C₁₋₆alkyl or —CH₂—O—C₁₋₄alkyl; with the proviso that when R₃ is alkoxy, R₁ is —C₁₋₆alkyl.

11. The compound or salt of claim 10, wherein R₂ is hydrogen.

12-20. (canceled)

21. The compound or salt of claim 1 for use as a vaccine adjuvant in treating an infectious disease in a human or animal.

22. The compound or salt of claim 2 for use as a vaccine adjuvant in treating an infectious disease in a human or animal.

23. A pharmaceutical composition comprising an effective amount of a compound or salt of claim 1 in combination with a pharmaceutically acceptable carrier.

24. The pharmaceutical composition of claim 23, further comprising an antigen.

25. The pharmaceutical composition of claim 23 for use in treating an infectious disease in a human or animal.

26. A method of treating a neoplastic disease in a human or animal by administering an effective amount of a compound or salt of Formula (I) 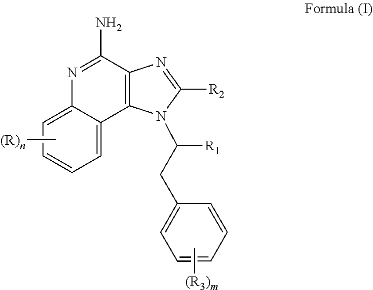
wherein:
m is an integer of 0 or 1;
n is an integer of 0 or 1;
R is selected from the group consisting of halogen, hydroxy, alkyl, alkoxy, and —C(O)—O-alkyl;
R₁ is alkyl or —CH₂—O—C₁₋₄alkyl;
R₂ is selected from the group consisting of hydrogen, methyl, ethyl, n-propyl, n-butyl, —CH₂OCH₃, —CH₂OCH₂CH₃, and —CH₂CH₂OCH₃; and
R₃ is selected from the group consisting of halogen, hydroxy, alkyl, and alkoxy; with the proviso that when R₃ is alkoxy, R₁ is alkyl.

27. The method of treating of claim 26, wherein the compound or salt is of Formula (II)

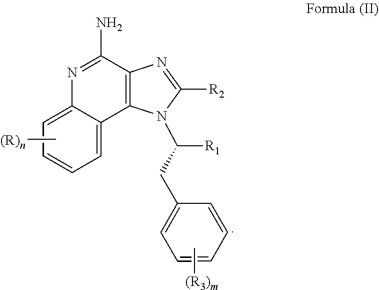

28. The method of treating of claim 26, wherein treating the neoplastic disease comprises inducing cytokine biosynthesis.
